(19) United States
(12) Patent Application Publication
(10) Pub. No.: US 2022/0088304 A1
(43) Pub. Date: Mar. 24, 2022

(54) SYSTEMS AND METHODS FOR CLOSED-LOOP CONTROL OF INSULIN-GLUCOSE DYNAMICS

(71) Applicant: PRESIDENT AND FELLOWS OF HARVARD COLLEGE, Cambridge, MA (US)

(72) Inventors: Eyal DASSAU, Boston, MA (US); Ankush CHAKRABARTY, Boston, MA (US); Francis J. DOYLE, III, Cambridge, MA (US)

(73) Assignee: PRESIDENT AND FELLOWS OF HARVARD COLLEGE, Cambridge, MA (US)

(21) Appl. No.: 17/298,417

(22) PCT Filed: Nov. 26, 2019
(86) PCT No.: PCT/US2019/063214

§ 371 Date: May 28, 2021

Related U.S. Application Data
(60) Provisional application No. 62/773,688, filed on Nov. 30, 2018.

(51) Int. Cl. A61M 5/172; A61B 5/00; A61B 5/145

(52) U.S. Cl. CPC A61M 5/1723; A61B 5/4839; A61M 2210/1017; A61M 2230/201; A61M 2005/1726; A61B 5/14532

(57) ABSTRACT

The present disclosure provides for systems and methods for maintaining glycemic control of a patient. An exemplary method can provide for first receiving glucose data from at least one sensor in an intraperitoneal space of the patient. The method can then provide for processing the received glucose data at a glucose monitoring system to yield processed data. The method can then provide for instructing, by the glucose monitoring system, an insulin infusion pump. Instructing the insulin infusion pump can be based on a closed-loop PID control algorithm and the processed data.

### CROSS-REFERENCE TO RELATED APPLICATIONS

This application claims priority to and the benefit of U.S. Provisional Patent Application No. 62/773,688, and titled “SYSTEMS AND METHODS FOR CLOSED-LOOP CONTROL OF INSULIN-GLUCOSE DYNAMICS,” filed Nov. 30, 2018, which is incorporated herein by reference in its entirety.

### STATEMENT REGARDING FEDERALLY SPONSORED RESEARCH

This invention was made with government support under DK101068 awarded by National Institutes of Health. The government has certain rights in the invention.

### FIELD

The present invention is directed to systems and methods for controlling insulin-glucose dynamics through closed-loop control.

### BACKGROUND

The following description includes information that may be useful in understanding the present invention. It is not an admission that any of the information provided herein is prior art or relevant to the presently claimed invention, or that any publication specifically or implicitly referenced is prior art.

Various feedback control strategies in state-of-the art single-hormone artificial pancreas (AP) systems are deemed clinically safe to regulate glucose in people with type 1 diabetes mellitus (T1DM). Current AP systems operate via subcutaneous (SC) glucose sensing and SC insulin delivery (SC-SC). Due to slow diffusion and transport dynamics across the interstitial space, even the most sophisticated control algorithms in on-body AP systems cannot react fast enough to maintain tight glycemic control under the effect of exogenous glucose disturbances caused by ingesting meals or performing physical activity. Additionally, transport delays in the interstitial fluid renders slow insulin absorption and slow insulin clearance. Furthermore, conventional methods for maintaining glycemic control of a patient rely on pure feedback control, where insulin is injected purely in response to detected low glucose levels in the patient. Pure feedback control in the SC-SC case can result in sustained high postprandial glucose levels due to the delays in insulin absorption. In other cases, an SC-SC method can result in deleteriously low glucose levels due to the slow clearance rates. Because of the danger of prolonged or repeated exposure to these states for a patient, SC-SC methods require “meal announcement”, where manual boluses must compensate for ingested meals, often without improving HbA1c levels. Even meal announcements cannot adequately compensate for the problems with SC-SC methods because of the tendencies to incorrectly estimate meal sizes or incorrectly identify the macronutrient content of meals.

There what is needed are systems and methods for maintaining glycemic control of a patient without experiencing the deleteriously low glucose levels or the postprandial glucose levels.

### SUMMARY

The present disclosure provides for a system for maintaining glycemic control of a patient. In this disclosure, a “patient” may be a subject with diabetes. The system can include at least one sensor, an insulin infusion pump, and a glucose monitoring system. The at least one sensor can detect glucose in an intraperitoneal space of the patient. The insulin infusion pump and inject insulin into the intraperitoneal space. The glucose monitoring system can be configured to carry out a series of steps. The steps can include receiving data from the at least one sensor. The data can be data on glucose levels as detected by the at least one sensor in the intraperitoneal space of the patient. The steps can then include sending instructions to the insulin infusion pump. These instructions can be based on a closed-loop general algorithm, such as a proportional-integral-derivative (PID) control algorithm, and the received data.

In some examples, the closed-loop PID control algorithm can include an optimization-based transfer function matching method.

In some examples, the closed-loop PID control algorithm can include a discrete-time transfer function model. In some examples, this discrete-time transfer function model can compensate for steady-state gain contributed by poles of the discrete-time transfer function model. In some examples, this discrete-time transfer function model can include a total daily insulin intake for the patient.

In some examples, the glucose monitoring system can interpolate the received data. Interpolating the received data can include using a piecewise Hermite or Legendre polynomial interpolation scheme.

In some examples, the glucose monitoring system can obtain at least one constant of the closed-loop PID control algorithm using discrete-time internal model control and optimization-based transfer function matching.

A second embodiment of the present disclosure can provide for a method for maintaining glycemic control of a patient. The method can include receiving glucose data from at least one sensor in an intraperitoneal space of the patient. The method can then provide for processing the received glucose data at a glucose monitoring system to yield processed data. The method can then provide for instructing, by the glucose monitoring system, an insulin infusion pump. Instructing the insulin infusion pump can be based on a closed-loop PID control algorithm and the processed data.

In some examples, the closed-loop PID control algorithm can include an optimization-based transfer function matching method.

In some examples, the closed-loop PID control algorithm can include a discrete-time transfer function model. In some examples, this discrete-time transfer function model can compensate for steady-state gain contributed by poles of the discrete-time transfer function model. In some examples, this discrete-time transfer function model can include a total daily insulin intake for the patient.

In some examples, the glucose monitoring system can interpolate the received data. Interpolating the received data can include using a piecewise Hermite or Legendre polynomial interpolation scheme.

In some examples, the glucose monitoring system can obtain at least one constant of the closed-loop PID control algorithm using discrete-time internal model control and optimization based transfer function matching.

A third embodiment of the present disclosure can provide for a non-transitory machine readable medium. The non-transitory machine readable medium can have stored instructions for performing a method. The instructions can include machine executable code which, when executed by at least one machine, causes the machine to perform a series of steps. The steps can include first receiving glucose data from at least one sensor. The steps can then provide for processing the received glucose data at a glucose monitoring system to yield processed data. The steps can then provide for instructing, by the glucose monitoring system, an insulin infusion pump, wherein the instructing is based on a closed-loop PID control algorithm and the processed data.

Additional details of the algorithm and the glucose monitoring system can be as provided above with respect to the first and second embodiment.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1A shows an exemplary glucose monitoring system, according to an embodiment of the present disclosure;

FIG. 1B shows an exemplary glucose monitoring system, according to an embodiment of the present disclosure;

FIG. 2 shows an exemplary methodology for maintaining glycemic control of a patient, according to an embodiment of the present disclosure;

FIGS. 3A-3D show exemplary fits between experimental data and an insulin-glucose dynamical model for raising a basal concentration of approximately 115 mg/dL up to 200 mg/dL, according to an embodiment of the present disclosure;

FIGS. 4A-4B show exemplary fits between experimental data and an insulin-glucose dynamical model for raising a basal concentration to 300 mg/dL, according to an embodiment of the present disclosure;

FIGS. 5A-5B show exemplary fits between experimental data and an insulin-glucose dynamical model for allowing plasma glucose to fall to 90 mg/dL, according to an embodiment of the present disclosure;

FIGS. 6A-6D show exemplary model validation data for the exemplary fits of FIGS. 3A-5B, according to an embodiment of the present disclosure;

FIG. 7 shows an exemplary impulse response comparison between conventional models of glycemic control and a model according to an embodiment of the present disclosure;

FIG. 8A shows an exemplary magnitude frequency domain characteristic comparison between conventional models of glycemic control and a model according to an embodiment of the present disclosure;

FIG. 8B shows an exemplary phase frequency domain characteristic comparison between conventional models of glycemic control and a model according to an embodiment of the present disclosure;

FIGS. 9A-9D show exemplary fits between experimental data and an insulin-glucose dynamical model for raising a basal concentration of approximately 115 mg/dL up to 200 mg/dL, according to an embodiment of the present disclosure;

FIGS. 10A-10B show exemplary fits between experimental data and an insulin-glucose dynamical model for raising a basal concentration to 300 mg/dL, according to an embodiment of the present disclosure;

FIGS. 11A-11B show exemplary fits between experimental data and an insulin-glucose dynamical model for allowing plasma glucose to fall to 90 mg/dL, according to an embodiment of the present disclosure;

FIG. 12 shows a protocol for testing an exemplary model for maintaining glycemic control according to an embodiment of the present disclosure;

FIG. 13A shows a comparison of a glucose trajectory, as compared between a conventional method for maintaining glycemic control and a closed-loop PID controller according to an embodiment of the present disclosure;

FIG. 13B shows a comparison of insulin delivery, as compared between a conventional method for maintaining glycemic control and a closed-loop PID controller according to an embodiment of the present disclosure;

FIG. 13C shows a comparison of a patient's time in a safe glycemic zone, as compared between a conventional method for maintaining glycemic control and a method according to an embodiment of the present disclosure;

FIG. 13D shows a comparison of a patient's time in hyperglycemia, as compared between a conventional method for maintaining glycemic control and a method according to an embodiment of the present disclosure;

FIG. 14A shows a comparison of a glucose trajectory, as compared between conventional methods for maintaining glycemic control and a closed-loop PID controller according to an embodiment of the present disclosure;

FIG. 14B shows a comparison of insulin delivery, as compared between conventional methods for maintaining glycemic control and a closed-loop PID controller according to an embodiment of the present disclosure;

FIG. 14C shows a comparison of a patient's time in hypoglycemia, as compared between conventional methods for maintaining glycemic control and a closed-loop PID controller according to an embodiment of the present disclosure;

FIG. 14D shows a comparison of a patient's time in a safe glycemic zone, as compared between conventional methods for maintaining glycemic control and a closed-loop PID controller according to an embodiment of the present disclosure;

FIG. 14E shows a comparison of a patient's time in hyperglycemia, as compared between conventional methods for maintaining glycemic control and a closed-loop PID controller according to an embodiment of the present disclosure;

FIG. 15A shows a blood glucose levels for an implementation of an exemplary PID controller on clinical data, according to an embodiment of the present disclosure;

FIG. 15B shows a comparison of the insulin delivery for a conventional method of maintaining glycemic control as compared against an exemplary PID controller, according to an embodiment of the present disclosure;

FIG. 16 shows an exemplary zoom-in view of the data in FIG. 5A, according to an embodiment of the present disclosure;

FIG. 17 shows an exemplary diagram for tuning a PID controller, according to an embodiment of the present disclosure;

FIG. 18A shows exemplary data of the magnitude of transfer function matching via a convex optimization, according to an embodiment of the present disclosure;

FIG. 18B shows exemplary data of the phase of transfer function matching via a convex optimization, according to an embodiment of the present disclosure; and

FIG. 19 shows an exemplary experimental protocol, according to an embodiment of the present disclosure.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Examples of using PIDs and MPCs are described in, for example, Huyett et al, Design and evaluation of a robust PID controller for a fully implantable artificial pancreas. Ind. Eng. Chem. Res. 54 10311-10321, 2015, which is incorporated by reference herein in its entirety.

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Overview

The present disclosure provides for determining the dynamic association between intraperitoneal (“IP”) insulin boluses and plasma glucose levels and constructing a new mathematical model based on this dynamic association. The present disclosure can therefore provide for a closed-loop control strategy to be deployed. An exemplary embodiment of the control strategy can be a transfer function leveraged by a PID controller with insulin feedback to prevent controller-induced hypoglycemia. Various embodiments of the present disclosure can provide for an artificial pancreas which senses glucose in the IP cavity and provides insulin injections into the IP cavity according to the sensed glucose. Although the present disclosure primarily discusses IP sensing and delivery, the present disclosure contemplates that such a system can also provide for sensing and delivering subcutaneously, or any other method as known in the art.

An exemplary controller, according to an embodiment of the present disclosure, can significantly outperform conventional controllers because the closed-loop system of the present disclosure better accounts for (1) delays and noise in a measurement signal (for example, when glucose is sensed subcutaneously) and (2) deleterious glycemic changes (such as sudden glucose decline due to physical activity). The present disclosure can generate a more effective control algorithm. Various systems and methods of the present disclosure can provide for a fully-automated and implantable artificial pancreas systems.

The present disclosure provides for systems and methods to detect glucose levels in the IP space and also inject insulin in the IP space. The IP space is a region within the abdominal cavity where the insulin-glucose kinetics are observed to be much more rapid than in the SC space. This faster insulin clearance in the IP cavity results not only in fewer hypoglycemic (glucose <70 mg/dL) episodes, but markedly improved postprandial glycemic regulation performance, compared to SC delivery, even without meal announcement. Furthermore, infusion in the IP cavity more closely approximates the body's natural insulin distribution, where insulin is maintained at a concentration 3-fold higher in the portal circulation than the peripheral circulation. This physiologic balance leads to less hypoglycemia and insulin resistance as well as improved glycemic variability compared with iatrogenic peripheral circulation hyperinsulinemia resulting from SC delivery. IP delivery also produces beneficial endocrine effects. For example, prolonged SC delivery adversely affects insulin-like growth factor-1 concentrations; this adversity is not observed in prolonged IP delivery. Therefore, systems and methods for maintaining glycemic control which rely on IP sensing and delivery can more closely approximate the body's natural insulin distribution.

Although the IP space offers clear benefits both from physiological and engineering perspectives, a fully implantable AP residing in the IP cavity, as provided for by the present disclosure, can reap the benefits of IP delivery and IP sensing. IP sensing can be significantly faster than SC sensing, and therefore, a controller according to an embodiment of the present disclosure can be (1) more reactive to glycemic disturbances and (2) more aggressive to maintain tight control with higher insulin clearance rates: this is especially advantageous for embedded/implantable AP technology where fast speeds and intermittent decision-making enable longer power cycles.

### Systems and Methods for Maintaining Glycemic Control

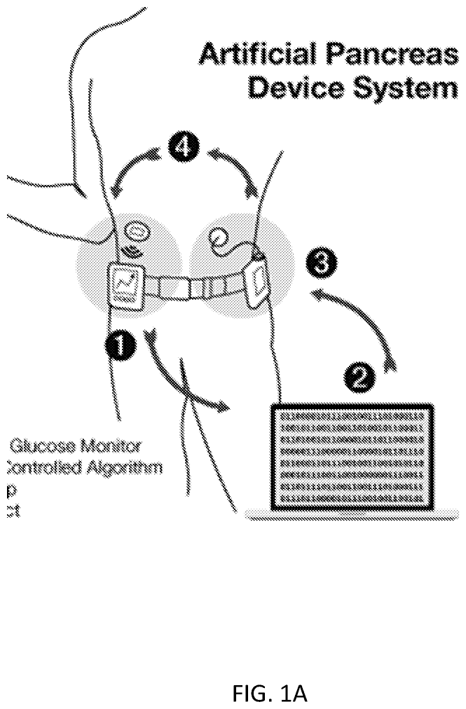

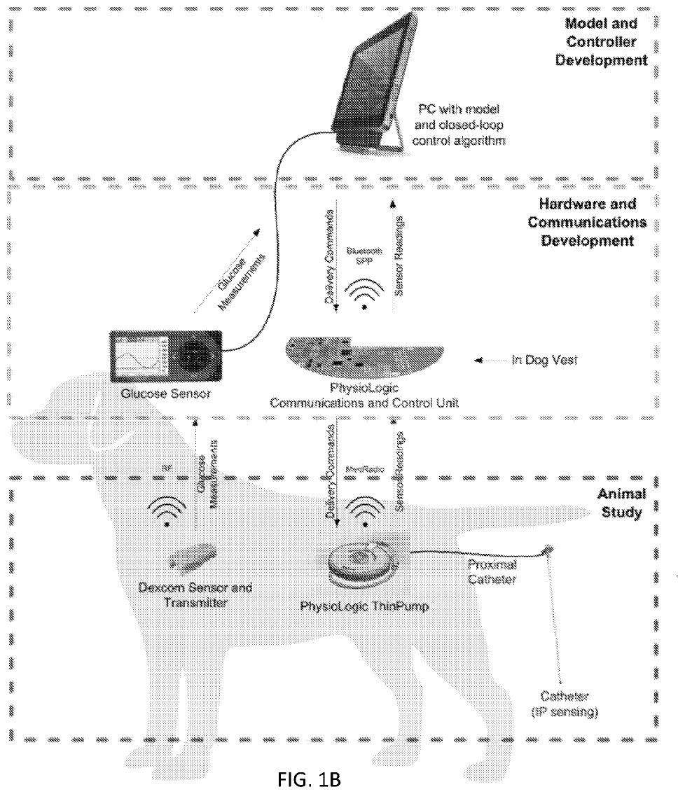

FIG. 1A shows an exemplary glucose monitoring system 100A for a patient, according to an embodiment of the present disclosure. System 100A can include a continuous glucose monitor 110; a sensor 112; an insulin delivery mechanism 114; an insulin pump 116; and an external controller 118.

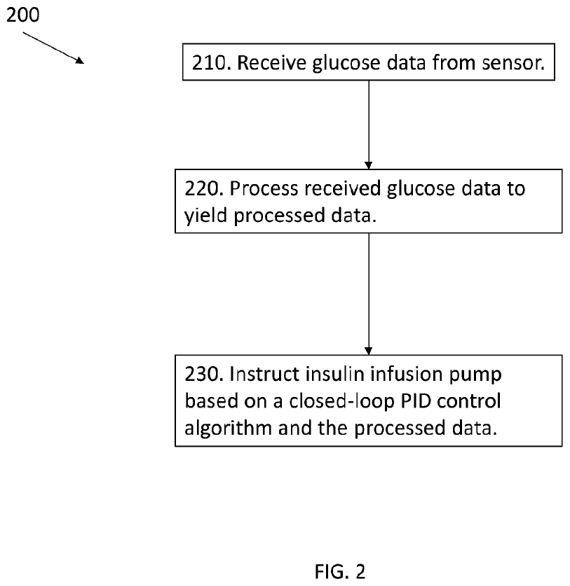

The continuous glucose monitor can receive glucose data from the sensor 112. The sensor 112 can be implanted in the IP space of the patient. The sensor 112 can transmit the glucose data to the continuous glucose monitor 110 via a transmitter (not pictured). In some examples, the continuous glucose monitor 110 can display an estimate of blood glucose levels, their direction, and a rate of change of these estimates. The continuous glucose monitor 110 can be further configured to communicate with and send the glucose data to an external controller 118. The external controller 118 can provide a control algorithm which outputs dosing instructions to an insulin pump 116. The insulin pump 116 can inject insulin into the IP space of the patient, based on the instructions from the external controller 118, via the insulin delivery mechanism 114. The external controller 118 can provide a control algorithm in accordance with the exemplary implementation discussed further with respect to FIG. 2.

Therefore, an exemplary glucose monitoring system 100A can provide a fully-autonomous, implantable artificial pancreas which can detect and respond to glucose levels in a patient. Additional features of system 100A, especially with regards to characteristics of the external controller 118, are discussed throughout the present disclosure.

FIG. 1B provides another exemplary embodiment of a glucose monitoring system 100B comprising different levels of device communication, according to an embodiment of the present disclosure. System 100B can include a remote computing system 130; a hardware and communications system 140; and a IP delivery system 150 in a patient. The remote computing system 130 can include a controller 132. The hardware and communication system 140 can include a glucose sensor 142 and a communications and control unit 144. The IP delivery system 150 can include a sensor and transmitter 152, an insulin pump 154, a proximal catheter 154, and an IP sensing catheter 158.

The controller 132 can provide for receiving glucose data from the glucose sensor 14. The controller 132 can then determine, according to a closed-loop algorithm (discussed further with regards to FIG. 2), when to instruct the communications and control unit 144 to inject insulin into the patient.

The glucose sensor 142 can be configured to send glucose measurements to the controller 132 and can be configured to receive glucose measurements from a sensor and transmitter 152. The communications and control unit 144 can receive sensor readings from the insulin pump 154. For example, the sensor readings can determine when insulin has been injected into the patient and how much insulin was injected. The communications and control unit 144 can transmit the sensor readings to the controller 132. The communications and control unit 144 can further receive instructions from the controller 132 on when to provide insulin to the patient. The communications and control unit 144 can send the instructions to the insulin pump 154 and consequently provide for injecting insulin into an IP space of the patient. The communications and control unit 144 can be, for example, a PhysioLogic Communication and Control Unit. In some examples of the present disclosure, the glucose sensor 142 and the communications and control unit 144 can be provided for in a pump, sensor, or wearable device (for example, a watch or phone). The communications and control unit 144 can hold an algorithm for instructing the insulin pump 154, as discussed further below with respect to FIG. 2.

The sensor and transmitter 152 can be imbedded in a patient, for example, in the IP space of a patient. The sensor and transmitter 152 can be in any SC space of a patient. In some examples, the sensor and transmitter 152 can be a Dexcomm sensor and transmitter. The sensor and transmitter 152 can communicate the glucose measurements to the glucose sensor 142. The insulin pump 154 can be implanted in a patient, for example in the IP space of a patient. The insulin pump 154 can be, for example, a Physiologic ThinPump. In some examples, the sensor and transmitter 152 can be a subcutaneous sensor and transmitter, or any other sensor and transmitter which is outside the IP space of a patient. Similarly, in some examples, the insulin pump can be a subcutaneous insulin pump, or any other insulin pump which is outside the IP space of a patient.

The insulin pump 154 can be configured to receive instructions from a communications and control unit 144 and be configured to send sensor readings to the communications and control unit 144. A IP sensing catheter 158 can be a sterile, 18-gauge nylon catheter, primed with U-400 insulin ex-vivo, advanced through the proximal catheter 154 such that the tip of the IP sensing catheter 158 extended 5 cm past the end of the proximal catheter 154 and into the IP space. The proximal catheter 154 can be an exteriorized IP access catheter.

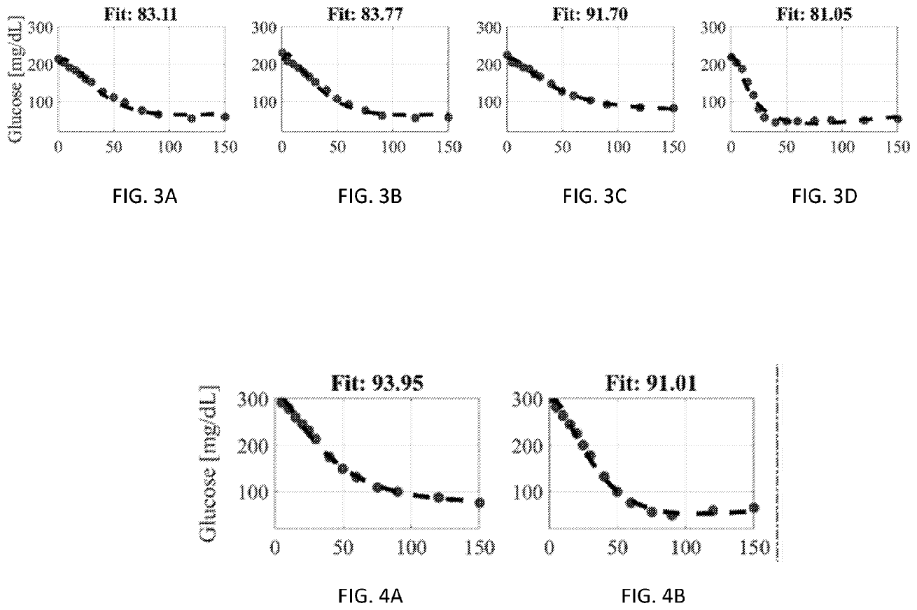

System 100B, for example, can be used to test the effectiveness of various embodiments of the present disclosure in an experimental design. For example, data collected for FIGS. 3A-6D, 9A-11B, and 18A-18B can be collected according to a system similar to system 100B. In some embodiments of the present disclosure, an exemplary system can comprise just a controller 132 and communications and control unit 144.

FIG. 2 shows an exemplary methodology 200 for maintaining glycemic control of a patient, according to an embodiment of the present disclosure.

The exemplary methodology 200 can begin at step 210 with receiving glucose data from a sensor. The sensor can be in the IP space of a patient, as discussed above with respect to FIGS. 1A-1B. Referring back to FIG. 2, in some examples of the present disclosure, the data received can be stored clinical data, experimental data, or test data. Therefore, step 210 can provide for sensing real-time measurements from a patient but can also receive stored data.

Methodology 200 can then proceed to step 220. At step 220, the methodology 200 can provide for processing the received glucose data of step 220 to yield processed data. In some examples of the present disclosure, data can be collected periodically in step 210, and step 220 can provide for interpolating the data at set intervals. For example, the data can be interpolated every 5 minutes (although the present disclosure provides for intervals of 5 minutes, other appropriate intervals can be provided for, such as every 1 second, 5 seconds, 10 seconds, 15 seconds, 30 seconds, 1 minute, 10 minutes, 15 minutes, 20 minutes, 30 minutes, or other appropriate intervals). The data can be interpolated via a piecewise Hermite polynomial interpolation scheme. The piecewise Hermite polynomial interpolation scheme provides shape-preserving properties during interpolation: that is, Hermite polynomials do not introduce unnecessary undulations to the underlying data to maintain continuous second-derivatives, unlike various other schemes like cubic splines. Constructing local linear models with respect to basal glucose magnitudes and basal insulin rates enable personalization of the model, since basal insulin rates differ widely within mammalian populations.

After processing the data, the methodology 200 can proceed to step 230, where the methodology 200 provides for instructing an insulin infusion pump based on a closed-loop general algorithm, such as a PID control algorithm, and the processed data. The instructions and communications between components, can be as provided for with respect to the systems 100A and 100B of FIGS. 1A and 1B, respectively. Step 230 can be completed by a remote, external controller, or by a controller within an artificial pancreas.

The instructions can be based on a closed-loop PID control algorithm and the processed data. As discussed with more detail below, the closed-loop PID control algorithm can be an optimization-based transfer function matching method. In some examples, the closed-loop PID control algorithm can be a discrete-time transfer function model. This discrete-time transfer function model can compensate for steady-state gain contributed by poles of the discrete-time transfer function model and can include a total daily insulin intake for the patient. The controller can obtain at least one constant of the closed-loop PID control algorithm using discrete-time internal model control and optimization-based transfer function matching.

Decision-making algorithms such as model predictive control and PID control typically require a mathematical model that captures the dynamics between insulin infusion and the corresponding glucose response. The present disclosure can utilize experimental glucose data obtained in real-time to enable formulation of control-relevant models for IP-IP dynamics.

Some examples of step 230 can provide for constructing a transfer function having the form:

$$H ( {z}^{- 1} ) = \frac{Y ( {z}^{- 1} )}{U ( {z}^{- 1} )} = \frac{{Kz}^{- 1}}{{\prod }_{j = 1}^{3} ( 1 - {p}_{j} {z}^{- 1} )} {Z}^{- L} . \qquad Equation 1$$

where z⁻¹ is a shift operator having a sampling time of 5 min, Y denotes the blood glucose deviation from the basal glucose in mg/dL, U is the insulin infusion rate in U/ 5 min, L ∈ N (the set of natural numbers) denotes an actuation lag in minutes, representing the diffusion lag exhibited by insulin at the site of infusion, K is a gain parameter, and p_{1,2,3} are the poles of the transfer function model. The model fitting procedure comprises of optimizing five parameters: namely, K, p₁, p₂, p₃, and L.

The present disclosure can provide for using normalized nonlinear least squares to compute the model parameters of Equation 1. This can be done, for example, in MATLAB R2016a via the tfest function, using the interpolated data from step 220. For example, data sampled every 5 minutes for a period of 150 minutes Y_{0.5:150} can have multiple values of L ∈ {0, 1, 2, 3}.

In order to validate the constructed model, the present disclosure can provide for using artificially constructed insulin and glucose data to augment the experimental insulin-glucose data (as discussed with respect to step 210). The validations of such data usage are discussed further with respect to FIGS. 3A-6D, 9A-11B, and 18A-18B.

Before instructing the insulin pump in step 230, the controller can provide for first tuning its gains. The controller can be a PID controller, where PID controllers typically comprise three design variables. Conventionally, it is challenging to compute these variables systematically for a wide range of systems while maintaining tight yet robust control performance. Classical tuning rules such as Ziegler-Nichols tuning tend to be very aggressive, which can result in controller-induced hypoglycemia. Alternatively, internal model control (IMC) based analytic tuning rules have exhibited good glucose regulation properties. The present disclosure provides for a digital control variant of the IMC tuning rules to obtain a PID controller in the incremental form:

uₖ=uₖ₋₁+ΔPₖ+ΔIₖ+ΔDₖ Equation 2.

where Equations 3-5 provide for the proportional, integral, and derivative components:

$$\begin{matrix}Δ {P}_{k} = {K}_{p} Δ {e}_{k} . & Equation 3 \\ Δ {I}_{k} = \frac{{K}_{p} {T}_{s}}{{T}_{i}} Δ {e}_{k} . & Equation 4 \\ Δ {D}_{k} = \frac{{K}_{p} {T}_{d}}{{T}_{s}} ( Δ {e}_{k} - Δ {e}_{k - 1} ) . & Equation 5\end{matrix}$$

where Equations 6-7 provide for additional descriptions of Equations 3-5:

Δeₖ=eₖ−eₖ₋₁ Equation 6.

eₖ=yₖ−rₖ Equation 7.

uₖ is the insulin bolus at time instant k, Tₛ is the sampling time of the system, yₖ is the glucose output, rₖ is a set-point to which the glucose is driven, Kₚ is the proportional gain, Tᵢ and T_{d} are integral and derivative time constants, respectively. Therefore, according to this method of tuning a PID controller, the present disclosure can provide for a systematic computation of the variables while maintaining an accurate and robust glycemic control which does not result in controller-induced hypoglycemia.

A transfer function equivalent in the z-domain for the PID with structure of Equation 1 is given by:

$${G}_{PID} ( {z}^{- 1} ) = \frac{u ( {z}^{- 1} )}{E ( {z}^{- 1} )} = \frac{{b}_{0} + {b}_{1} {z}^{- 1} + {b}_{2} {z}^{- 2}}{1 - {z}^{- 1}} . \qquad Equation 8$$

where U(z⁻¹) and E(z⁻¹) are the z-transforms of the control action and error signals, respectively, and

$${b}_{0} = {K}_{p} ( 1 + \frac{{T}_{s}}{{T}_{i}} + \frac{{T}_{d}}{{T}_{s}} ) , {b}_{1} = - {K}_{p} ( 1 + \frac{2 {T}_{d}}{{T}_{s}} ) , {b}_{2} = \frac{{K}_{p} {T}_{d}}{{T}_{s}} . \qquad Equation 9$$

The present disclosure provides a method for obtaining the constants b₀, b₁ and b₂ (and hence the controller gains) using discrete-time IMC and optimization-based transfer function matching. The present disclosure can provide for first factorizing H(z⁻¹) into two components: an all-pass component Ha(z⁻¹)=z⁻³ and a minimum-phase component as shown in Equation 10:

$$\tilde{H} ( {z}^{- 1} ) = \frac{1800}{TDI} \frac{{K}_{s} {K}_{m}}{( 1 - 0.61 {z}^{- 1} ) {( 1 - 0.90 {z}^{- 1} )}^{2}} . \qquad Equation 10$$

Then the present disclosure can provide for computing G_{IMC} as in Equation 11:

G_{IMC}(z⁻¹)=G_{q}(z⁻¹)G_{f}(z⁻¹) Equation 11.

where:

G_{q}(z⁻¹)=(z^{-q}\tilde{H}(z⁻¹))⁻¹ Equation 12.

has a design parameter q ∈ N that ensures Gq(z⁻¹) has numerator and denominator polynomials if identical degree in z⁻¹, and:

$${G}_{f} ( {z}^{- 1} ) = \frac{1 - λ}{1 - λ {z}^{- 1}} . \qquad Equation 13$$

Equation 13 provides a low-pass filter with a tuning parameter λϵ (0, 1) that enables a user to trade-off set-point tracking (λ→0) and disturbance rejection (λ→1). In some examples of the present disclosure, λ=0.98 since a primary concern is rejecting glucose disturbances. Therefore:

$${\tilde{G}}_{PID} ( {z}^{- 1} ) = \frac{{G}_{IMC} ( {z}^{- 1} )}{1 - H ( {z}^{- 1} ) {G}_{IMC} ( {z}^{- 1} )} . \qquad Equation 14$$

Equation 14 can be provided to deal with control-relevant models of order ≥3.

Substituting Equation 11 into Equation 14 generally yields transfer functions of order higher than that described in Equation 8. While many methods have been proposed in the literature for dealing with specific classes of H(z⁻¹) that yields simple forms of \tilde{G}_{PID}(z⁻¹), the present disclosure can therefore provide an optimization-based procedure to compute b0, b1, and b2 by matching the transfer functions G_{PID} and \tilde{G}_{PID}. The present disclosure can sample Ns points in the frequency domain within a closed-loop bandwidth (for example, 1.2×10−2, 8.0×10−2 rad/min). The present disclosure can require Equation 8 to resemble Equation 14 most closely. This enables the present disclosure to provide the following optimization problem in Equation 15:

$$arg {min}_{{b}_{0} , {b}_{1} , {b}_{2}} {\sum }_{ℓ = 1}^{{N}_{s}} ( {w}_{1} {δ}_{{m}_{ℓ}}^{2} + {w}_{2} {δ}_{{p}_{ℓ}}^{2} ) . \qquad Equation 15$$

subject to the following definitions in Equations 16-19.

[symbol]

=|G_{PID}( [symbol] )|−|\tilde{G}_{PID}(wₗ)|, Equation 16.

[symbol]

=∠G_{PID}(wₗ)−∠\tilde{G}_{PID}(wₗ) Equation 17.

G_{PID}(wₗ)=G_{PID}(z⁻¹)|_{z}_{^{−1}}₌ₑₓₚ₍₋ [symbol] _{T}_{ₛ}₎, Equation 18.

\tilde{G}_{PID}(wₗ)=\tilde{G}_{PID}(z⁻¹)|_{z}_{^{−1}}₌ₑₓₚ₍₋ [symbol] _{T}_{ₛ}₎, Equation 19.

where i=\sqrt{−1}. Equation 15 can be solved using any constrained optimization solver. In some examples, Equation 15 can be solved using a Nelder-Mead simplex procedure (MATLAB: fminsearch) with w₁=1 and w₂=0.01 yields optimal coefficients b₀*, b₁*, b₂* from which the present disclosure can obtain Kₚ, Tᵢ, and T_{d} using Equation 9. Specifically, the present disclosure can provide for:

Kₚ=−9.18×10⁻⁵×TID[U/5 min] Tᵢ=76.94 min T_{d}=33.38 min

Obtaining Kₚ, Tᵢ, and T_{d} as such can be used for the design of a PID in any other application where the dynamics can be described via transfer function models.

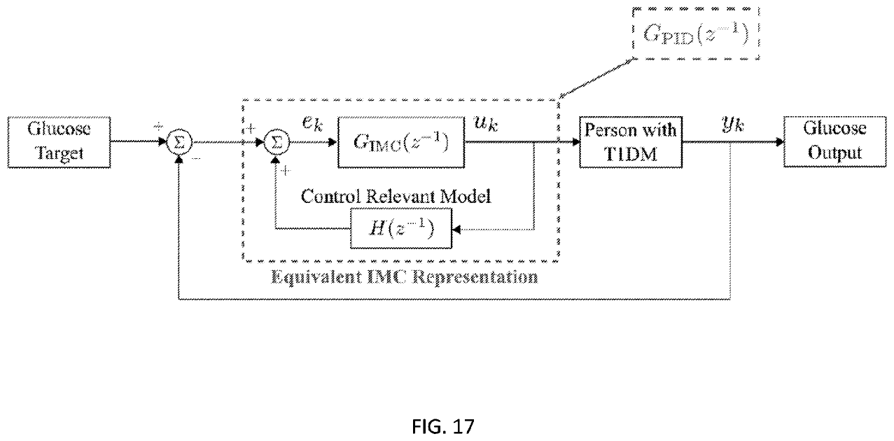

FIG. 17 shows an exemplary diagram for tuning a PID controller, according to the method of developing a control model shown with regards to providing the optimization problem described in Equations 15 to 19. The present disclosure can also provide for modifying the nominal PID controller structure described in Equation 2 in order to provide immunity against adverse effects occurring in practice such as derivative kick (sharp jump in control actions caused by changing glucose targets) or integral windup (large control actions due to accumulation of non-zero tracking error). In some embodiments, protecting against derivative kick is implemented using a low-pass filtering of the derivative gain term via

$$Δ {D}_{k} = \frac{β {T}_{d}}{{T}_{s} + β {T}_{d}} Δ {D}_{k - 1} + {K}_{p} \frac{{T}_{d}}{{T}_{s} + β {T}_{d}} ( Δ {e}_{k} - Δ {e}_{k - 1} ) . \qquad Equation 20$$

where β=0.1 is a recommended derivative filter gain, and the integral windup is implemented using a forgetting factor 0<α<<1, where

$$Δ {I}_{k} = {K}_{p} exp ( - α \vert {e}_{k} \vert ) \frac{{T}_{s}}{{T}_{i}} . \qquad Equation 21$$

which ensures the integral term remains small in spite of high tracking error (such as after a glycemic disturbance), but rises during small magnitude but sustained error signals, thereby resulting in faster set-point tracking; for example, an embodiment of the present disclosure can choose α=0.04.

The present disclosure can further provide for predicting plasma insulin content and informing the instructing of the insulin infusion pump (step 230) based on these predictions. High quantities of insulin in the blood inhibits insulin production in a healthy β-cell. The present disclosure can provide for an insulin feedback (IFB) term that reduces the magnitude of the control obtained from the PID Equation 2. That is,

Uₖ=(1+γ)uₖ−γI_{P,k} Equation 22.

where Uₖ is the insulin actually infused into the patient in U/hr, I_{p,k} is the plasma insulin concentration predicted at time k in μU/mL, and γ is a positive scalar selected to express the degree to which the current insulin bolus is suppressed by plasma insulin. The scalar γ must be selected to ensure that Uₖ is equal to the basal insulin infusion rate when the system is in steady-state.

In order to construct a model for the plasma insulin estimate, the present disclosure can provide for employing the following continuous-time bi-exponential impulse response structure:

$${I}_{P} ( t ) = \frac{U ( t )}{{K}_{IFB} ( {τ}_{4} - {τ}_{3} )} ( {e}^{- \frac{t}{{τ}_{4}}} - {e}^{- \frac{t}{{τ}_{3}}} ) . \qquad Equation 23$$

where τ₃ and τ₄ are time constants in minutes. The interpolated data, as provided for in step 220, can obtain a richer dataset with 5-minute sampling time using Hermite polynomials, and employ a nonlinear least squares cost function (MATLAB: lsqcurvefit) to fit the model and validate on data constructed as in the insulin-glucose fitting procedure.

The discrete-time transfer function model

$$\overline{H} ( {z}^{- 1} ) = \frac{- 8.10 {z}^{- 3}}{( 1 - 0.61 {z}^{- 1} ) {( 1 - 0.90 {z}^{- 1} )}^{2}} . \qquad Equation 24$$

is found to provide the best fit based on the following goodness-of-fit metric:

$${J}_{fit} = 100 \frac{1 - \vert {\hat{Y}}_{0 \text{:} 5 \text{:} 150} - {Y}_{0 \text{:} 5 \text{:} 150} \vert}{\vert {\hat{Y}}_{0 \text{:} 5 \text{:} 150} - 𝔼 ( {Y}_{0 \text{:} 5 \text{:} 150} ) \vert} . \qquad Equation 25$$

where Ŷ denotes model predictions with the same input sequence as Y, and [symbol] (Y_{0:5:150}) represents the average of the interpolated training data sequence (more details are provided in the Methods section). Exemplary experimental data with corresponding model responses and goodness-of-fit metrics I_{fit} is provided further with respect to FIGS. 3A-6D, 9A-11B, and 18A-18B.

In some examples of the present disclosure, the model structure discussed above with respect to Equations 1-25 can be personalized for human testing. For example, the present disclosure contemplates the following patient-specific model

$$H ( {z}^{- 1} ) = \frac{1800}{TDI} \frac{{K}_{s} {K}_{m} {z}^{- 3}}{( 1 - 0.61 {z}^{- 1} ) {( 1 - 0.90 {z}^{- 1} )}^{2}} . \qquad Equation 26$$

where Ks is a unit-less safety factor that compensates for model mismatch, the scalar

Kₘ=−60(1−p₁)(1−p₂)(1−p₃) mg/dL/hr

is a constant for compensating the steady-state gain contributed by the poles of the transfer function, and TDI is the total daily insulin intake for the individual in question; this is the factor that enables personalization, along with the poles. The ‘1800’ factor arises from an empirical rule relating fall in blood glucose to insulin dose. This patient-specific model can provide highly accurate and personal results for each patient.

The present disclosure can provide for obtaining certain variables from experimental data. For example, plasma insulin data can be collected from an animal study (as discussed further below with respect to the Experimental Protocol), and the following statistics can be obtained: K_{IFB}=0.98±0.42 L/min, τ₃=34.51±12.88 min, and τ₄=14.87±4.88 min for the plasma insulin prediction model described in Equation 23. These values closely resemble conventional values, specifically 34.60±5.90 min, 17.40±4.70 min for the time constants τ₃ and τ₄, and 1 L/min for the gain K_{IFB}.

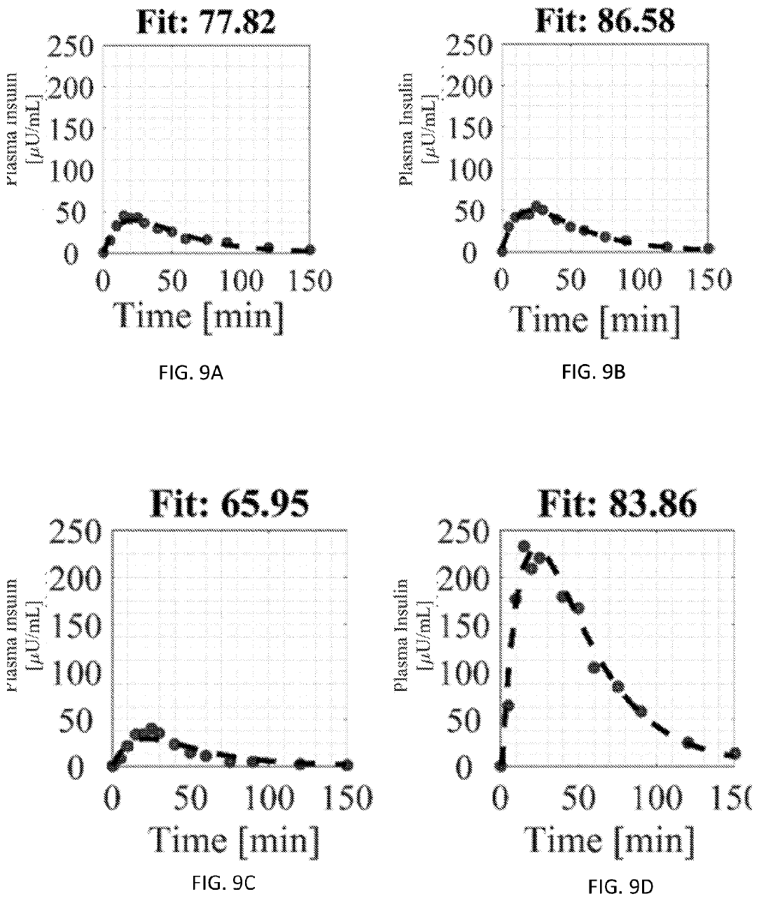

The model fits with corresponding fit [%] is provided in FIGS. 9A-11B. The fit of the validation data ranged between 49.2% and 92.1%. The plasma insulin model

$$\frac{{I}_{P} ( s )}{U ( s )} = \frac{1}{{K}_{IFB}} \frac{1}{( {τ}_{3} s + 1 ) ( {τ}_{4} s + 1 )}$$

is formulated with the average values obtained above, that is, τ₃=34.51 min, τ₄=14.87 min, and K_{IFB}=0.98 L/min. For discrete-time implementation with a 5 min sampling time, the plasma insulin estimate at time k is given by

I_{P,k}=1.58I_{P,k-1}−0.62I_{P,k-2}+0.02Uₖ₋₁+0.18Uₖ₋₂

which informs the controller through the Equation 22.

Therefore, FIGS. 1A-2 provide systems and methods to model insulin-glucose dynamics in the mammalian IP space and subsequently design a closed-loop control system for a fully autonomous, implantable AP using experimental data obtained from insulin impulse response tests on diabetic dogs.

Based on the calculations and measurements discussed above with respect to method 200, the present disclosure can therefore provide for employing model-fitting techniques to formulate a control-relevant discrete-time linear mathematical model of insulin effects and glycemic responses for an implantable AP. The present disclosure can also provide for constructing personalized dynamical models for human patients, based on total daily intake (TDI) of insulin, as reported in the literature. Although this dynamical model can be employed to design a wide range of control algorithms (such as model-based predictive controllers used in AP research), the present disclosure formulates a proportional-integral-derivative (PID) controller operating in the IP space, using a novel optimization-based transfer function matching method. Using a PID controller for the implantable AP allows for time delays between sensing and delivery have smaller magnitude in the IP space. A PID controller, according to an embodiment of the present disclosure, can also make use of faster diffusion dynamics in the IP space. Therefore, PID control performs similarly to computationally complex algorithms requiring higher memory capacity and execution times such as model predictive control (MPC). The present disclosure also provides for modeling the plasma insulin concentration based on experimental data in order to derive estimates of plasma insulin based on insulin dosing history. This model is exploited to derive an insulin feedback (IFB) term for the IP system.

### Experimental Protocol

The present disclosure provides for a transfer function model of insulin-glucose dynamics in the IP space constructed from data obtained by performing experiments on dogs exhibiting dynamics similar to humans with T1DM. FIGS. 3A-18B present experimental data obtained by various embodiments of the experimental protocol. These FIGS. 3A-18B show the success of an exemplary controller operating according to the various embodiments of the present disclosure.

All statistical analyses were performed in MATLAB R2016a using the Statistics and Machine Learning toolbox. All data are reported as means±one standard deviation or medians (interquartile ranges) as indicated. Comparison between two groups was performed using a two-tailed criteria Wilcoxon ranked sum test and significance determined at p<0.05 or p<0.001.

An exemplary experimental study can study three conscious adult mongrel dogs weighing 22-25 kg. The dogs can be fed a 65-75 kcal/kg/day diet of canned meat and chow (28% protein, 49% carbohydrate, and 23% fat). Two weeks prior to an experiment, the animals can be placed under general anesthesia and a catheter can be surgically placed in a femoral artery. A laparotomy can be performed for the placement of blood sampling catheters in the hepatic portal vein and hepatic vein. In addition, to provide access to the IP space during experiments, a silastic, polytetrafluoroethylenelined “guide” catheter can be placed within the lower right quadrant of the IP space. The free ends of the blood sampling and IP access catheters can be filled with a heparin/saline solution, knotted, and placed into respective subcutaneous pockets. All surgical incision sites can be closed; the dogs can be anesthetically recovered and permitted a minimum of 14 recovery days. Prior to study, each dog's health can be confirmed, evidenced by a leukocyte count <18,000/mm³, hematocrit >35%, good appetite, normal stooling, and healthy physical appearance. Therefore, an exemplary study can use system 100B as shown in FIG. 1B.

An exemplary embodiment of the present disclosure can provide for conducting experiments on the animals to determine appropriate constants for a model, according to methodology 200.

Animals can be fasted overnight prior to each experiment. On the morning of a study, the free ends of the IP access catheter and the blood sampling catheters were exteriorized from their subcutaneous pocket under local anesthesia (2% lidocaine). The dogs were placed in a Pavlov harness for the remainder of each experiment. Three protocols can be employed (as discussed further below).

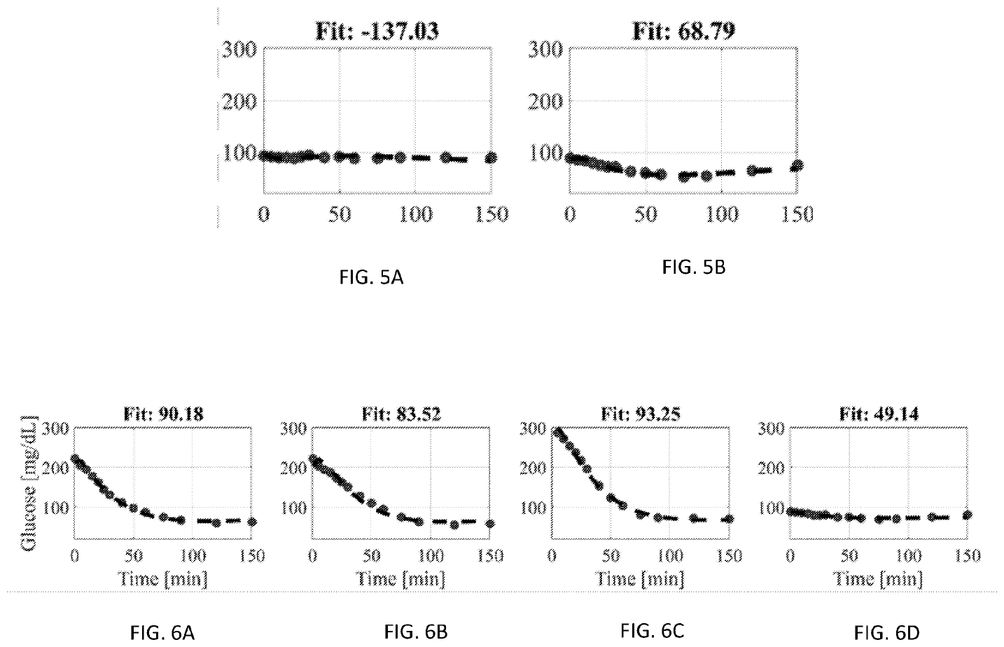

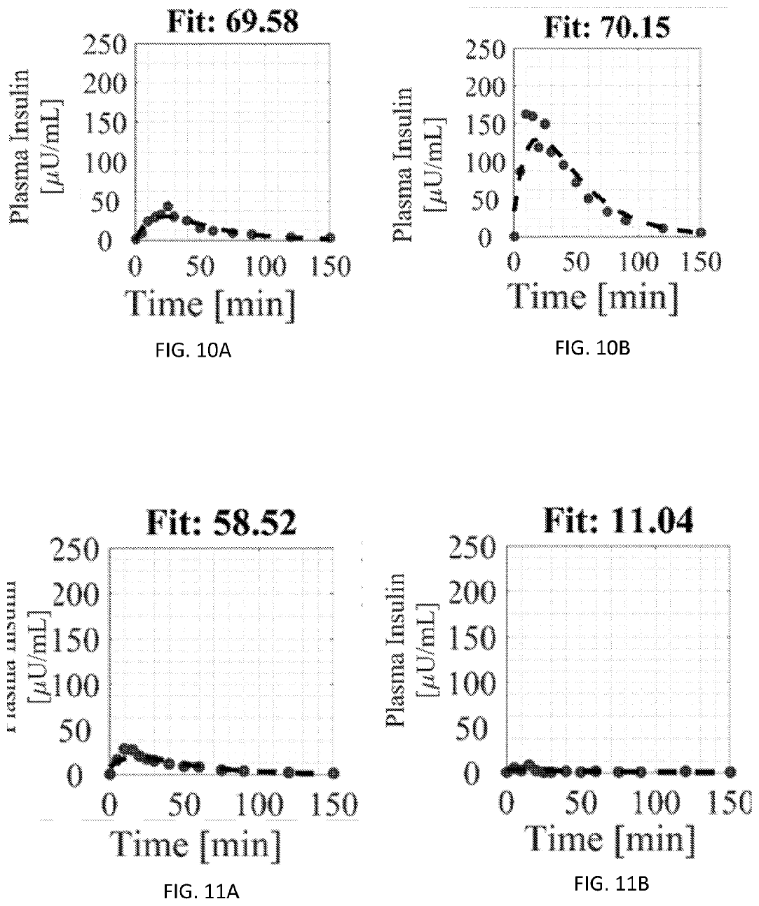

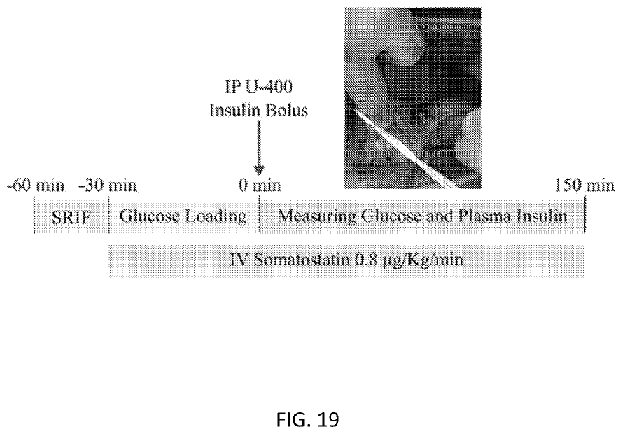

FIG. 19 shows an exemplary protocol for each experiment 1900. Each experiment 1900 can consist of a 30 min (Time 1) somatostatin equilibration period (1910), a 30 min (Time 2) glucose loading period (1912), an IP insulin bolus (1914) at Time 3, and a 150-minute (Time 4) glucose and insulin sampling period (1916). During the IP insulin bolus (1914) and the glucose and insulin sampling period (1916), IV Somatostatin can be infused intravenously at 0.8 μg/kg/min to inhibit endogenous insulin and glucagon secretion, approximating the insulin-deficient state of type 1 diabetes. Somatostatin rapidly and potently inhibits insulin production such that virtually all insulin in the plasma and interstitium was of exogenous origin by the time the bolus is given at 0 min. During the 30 min (Time 2) glucose loading period (1912), an intravenous infusion of 20% dextrose can be used to raise the arterial plasma glucose concentration from a basal concentration of approximately 115 mg/dL up to 200 mg/dL (protocol 1, n=4 experiments, results shown in FIGS. 3A-3D and 9A-9D), 300 mg/dL (protocol 2, n=2 experiments, results shown in FIGS. 4A-4B and 10A-10B), or no glucose was infused, allowing plasma glucose to fall to 90 mg/dL (protocol 3, n=2 experiments, results shown in FIGS. 5A-5B and 11A-11B); this can be observed to be the steady-state glucose level at euglycemia for the dogs.

Near the end of the 30 min (Time 2) glucose loading period (1912), a sterile, 18-gauge nylon catheter (Access Technologies, Skokie, Ill.) can be primed with U-400 insulin (Thermalin Diabetes, Cleveland, Ohio) ex-vivo, then advanced through the exteriorized IP access catheter such that the tip of the catheter extends 5 cm past the end of the guide catheter and into the IP space.

Simultaneously, at the end of the 30 min (Time 2) glucose loading period (1912), an IP insulin bolus can be using a 25 or 50 μL Hamilton syringe. In protocol 1, one dog was studied four times and received IP insulin boluses of 0.075, 0.15, 0.3, and 0.6 U/kg when arterial plasma glucose was ≈200 mg/dL. A second and third dog were studied twice, receiving 0.15 and 0.45 U/kg IP insulin doses in protocol 2 from an initial glucose level of approximately 300 mg/dL, and 0 and 0.075 U/kg in protocol 3 from an initial glucose level of 90 mg/dL. After the IP insulin bolus, blood was serially sampled for 150 min from the femoral artery, hepatic portal vein, and the hepatic vein; plasma glucose and insulin concentrations were determined. Following each study, the free ends of the catheters were placed into new subcutaneous pockets under general anesthesia for use in subsequent studies. All studies were conducted at least one week apart.

Although various timings are discussed with respect to FIGS. 3A-5B and 9A-11B, the present disclosure contemplates that other timings can be used for an experimental protocol, as would be readily understood by a person skilled in the art.

The canine model can study insulin-glucose kinetics and successfully evaluate closed-loop PID control algorithms with subcutaneous insulin delivery. Therefore, the present disclosure contemplates that the canine model can be used to study closed-loop PID control algorithms in the IP space because these kinetics closely resemble human glucose metabolism, insulin responsiveness, and absorption profiles in the IP space. Additionally, the animal's size and anatomical characteristics permit the placement of blood sampling catheters necessary or assessing the pharmacokinetics and pharmacodynamics in-vivo.

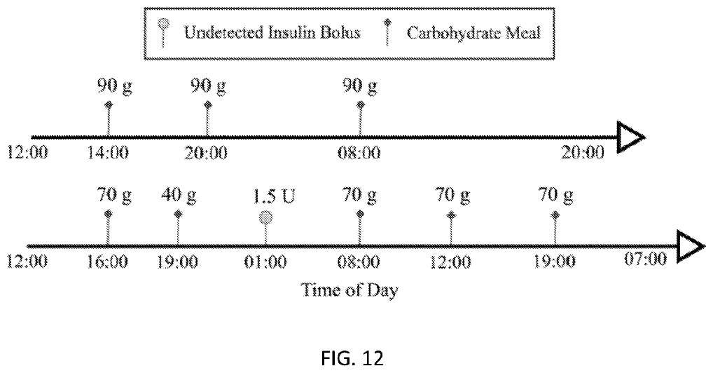

The experimental data section below provides results of simulation studies on the 10 patient cohort of the US FDA-accepted UVA/Padova simulator. Clinical scenarios for both nominal and robust analysis are illustrated further in FIG. 12. In the nominal scenario, three large (90 g CHO) meals are provided at widely spaced intervals to test set-point tracking of the proposed controller. In the robustness test scenario, five meals in total are consumed within 43 hours of closed-loop control. A 70 g meal of carbohydrates is given at 4 PM, after 4 hours of closed-loop initiation. A snack is provided at 7 PM containing 40 g CHO. At 1 AM, the safety of the closed-loop system is assessed by testing the controller's reaction to an undetected insulin bolus (representing manual overbolusing, latent exercise effects, or heightened insulin sensitivity due to illness). The next morning, a 70 g CHO breakfast is consumed at 8 AM, followed by a 70 g CHO lunch at noon, and a dinner of 70 g CHO at 7 PM. Meals in both scenarios are completely unannounced, that is, the controller is responsible for autonomously recommending appropriate insulin doses.

### Experimental Data—Animal Testing

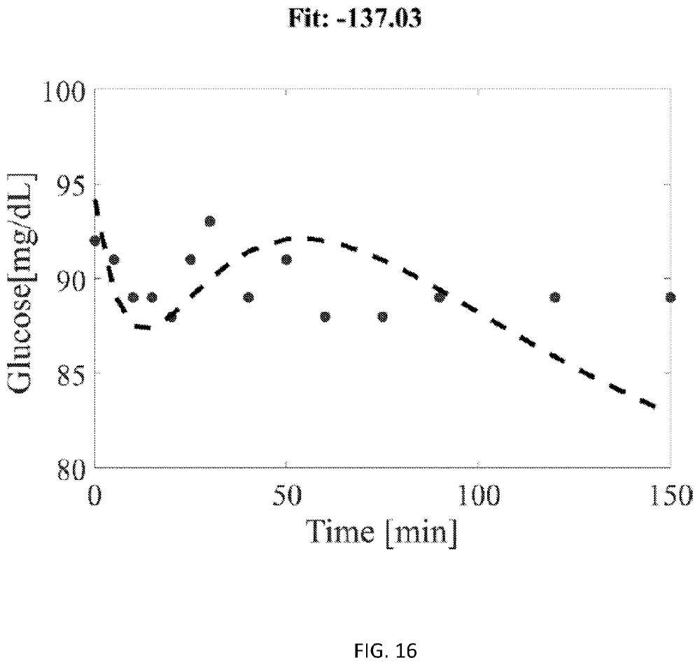

FIGS. 3A-3D show exemplary fits between experimental data and an insulin-glucose dynamical model for the first protocol; FIGS. 4A-4B show exemplary fits for the second protocol; and FIGS. 5A-5B show exemplary fits for the third protocol. The dots show experimental data of glucose obtained from the IP cavity and the dashed lines show the corresponding insulin-glucose dynamical model fit, according to an embodiment of the present disclosure. The fit percentages above each plot show the goodness of fit. FIG. 16 shows a zoom-in of the data for FIG. 5A.

FIGS. 9A-9D show exemplary fits between experimental data and an insulin-glucose dynamical model for the first protocol; FIGS. 10A-10B show exemplary fits for the second protocol; and FIGS. 11A-11B show exemplary fits for the third protocol. These graphs are based on the goodness of fit for experimental data and the patient-specific model, discussed above with respect to Equation 26. Similarly to the previous set of FIGS., 3A-3D, 4A-4B, and 5A-5B, the dots show experimental data of glucose obtained from the IP cavity and the dashed lines show the corresponding insulin-glucose dynamical model fit, according to an embodiment of the present disclosure.

For the impulse response tests in protocols 1 and 2, high model fits are achieved, ranging from 81% and 94%. The fits for protocol 3 are notably lower, with one fit being highly negative, in spite of the trend being suitably captured by the model (1) (see FIG. 16). This is explained as follows: for both experiments in protocol 3, the insulin infused is small (in one case it is zero), thus, the glucose variation is not large. Thus, small measurement errors result in highly oscillatory signals around a slowly-varying glucose signal: this noisy variability cannot be captured by a linear model, resulting in (seemingly) poor fits for this protocol. The response and kinetics of an exemplary model of the present disclosure, as described in Equation 24, can be compared with other transfer function models previously described in the literature: for SC-SC dynamics, and IP-IP dynamics.

FIGS. 6A-6D show exemplary model validation data for the exemplary fits of FIGS. 3A-5B, according to an embodiment of the present disclosure.

In order to validate the constructed models, the present disclosure contemplates using artificially constructed insulin and glucose data to augment experimental insulin-glucose data. Four data vectors can be generated for validation: the mean and median of the protocol 1 data (FIGS. 6A and 6B, respectively), and the mean of the protocol 2 and 3 data (FIGS. 6C and 6D, respectively). The proposed model demonstrates good performance on this validation set: the model validation performance is illustrated in the lowest block in FIG. 1B.

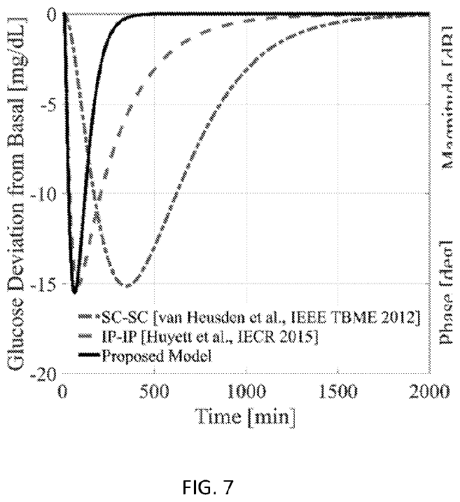

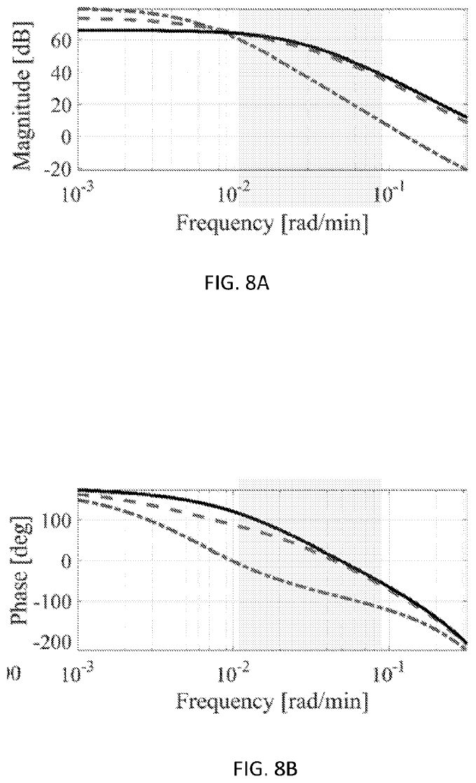

FIG. 7 shows an exemplary impulse response comparison between conventional models of glycemic control and a model according to an embodiment of the present disclosure. FIG. 8A shows an exemplary magnitude frequency domain characteristic comparison between conventional models of glycemic control and a model according to an embodiment of the present disclosure. FIG. 8B shows an exemplary phase frequency domain characteristic comparison between conventional models of glycemic control and a model according to an embodiment of the present disclosure.

As expected from the faster poles of the proposed model (0.61 vs. 0.75 in the previous model), FIG. 7 shows that the peak glucose deviation occurs around 1 hour in an impulse response test, as compared to the much slower peak time of 5 hours for the SC-SC model. Although the time to peak is similar with the proposed model and a conventional IP-IP model, the time needed to return to steady-state for the glucose is much faster in the proposed model, reflecting a faster clearance rate of insulin than that expected in the earlier model. Despite this disparity, the frequency characteristics of our model aligns closely to those described in the literature, especially around the estimated closed-loop bandwidth of 1.2×10⁻² rad/min and 8.0×10−2 rad/min, reported in those studies (see shaded area in FIGS. 8A-8B).

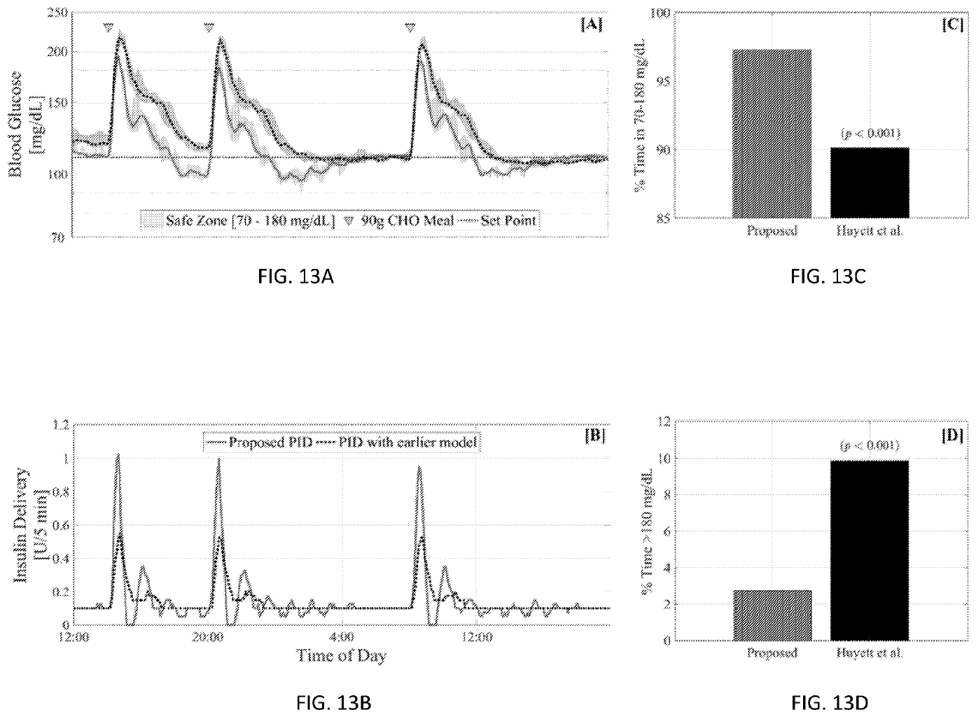

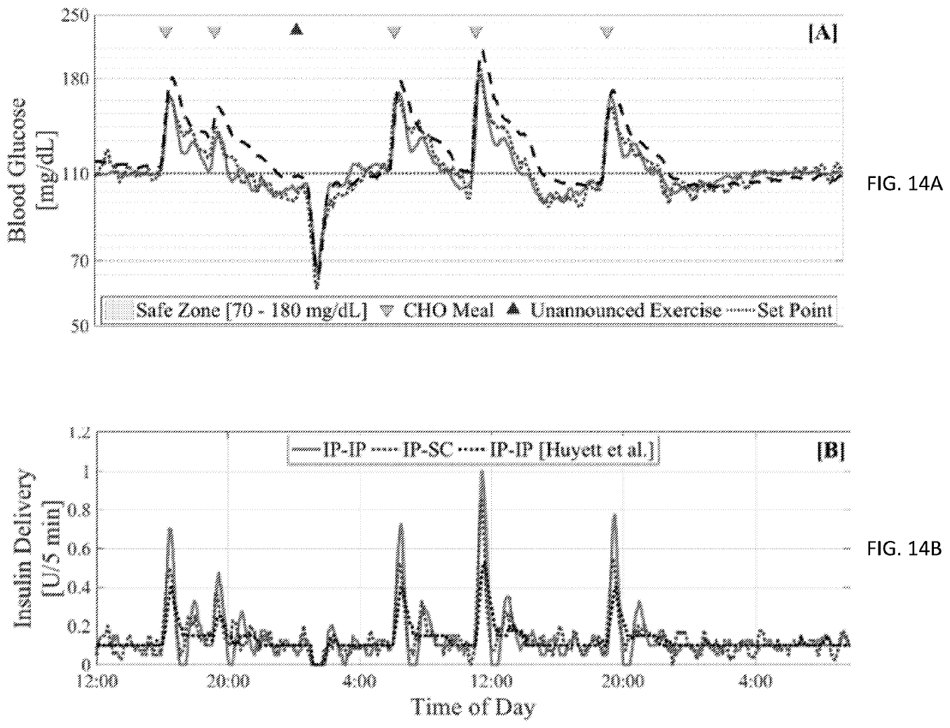

The simulation results of the comparative study are provided in FIGS. 13A-D, with corresponding glucose metrics reported below in Table 1. FIG. 13A shows that the glucose trajectory with the proposed controller (the solid line) is considerably tighter within the safe glycemic zone of 70-180 mg/dL compared to the corresponding glucose trajectory (dashed) based on the earlier model. As observed in FIG. 13B, the proposed controller is more aggressive than the conventional design, which is justified because the impulse response based on experimental data demonstrates higher insulin sensitivity and faster insulin clearance than the older model (see FIGS. 7-8B).

The beneficial effects of the present disclosure can be seen clearly from the clinical metrics presented in subplots FIGS. 13A-13B. The proposed PID results in significantly higher time in the 70-180 mg/dL range (97.3±1.5% vs. 90.1±5.6%; p<0.001) and lower time above 180 mg/dL (2.7±1.5% vs. 9.8±5.6%; p<0.001). It is also important to note that the proposed PID greatly outperforms the prior design in terms of time in the tight range of 80-120 mg/dL (73.0±5.9% vs. 54.8±8.7%; p<0.001). Additional glucose metrics are provided in Table 1 (second and third columns): the proposed PID not only improves time in range, but the median blood glucose (averaged over 10 patients) in spite of large unannounced meals is kept around 110 mg/dL with a very small standard deviation, unlike the prior controller (110.4±0.6 mg/dL vs. 117.5±4.9 mg/dL; p<0.001). Another positive consequence of such improved control performance is that the maximum blood glucose level is curtailed (196.4±9.8 mg/dL vs. 221.0±12.1 mg/dL; p<0.001).

TABLE 1
Glucose Metrics

| Clinical | Nominal | | | | |
|---|---|---|---|---|---|
| Metrics | Performance | | Robustness Analysis | | |
| Controller | Proposed | Earlier | Proposed | Proposed | Earlier |
| Type | PID | PID | PID | PID | PID |
| Insulin/ | IP/IP | IP/IP | IP/IP | IP/SC | IP/IP |
| Glucose | | | | | |
| BG < 54 | 0.00 ± | 0.00 ± | 0.00 ± | 0.53 ± | 0.13 ± |
| mg/dL [%] | 0.00 | 0.00 | 0.29 | 0.71 | 0.40 |
| BG < 70 | 0.00 ± | 0.00 ± | 0.69 ± | 1.25 ± | 0.67 ± |
| mg/dL [%] | 0.00 | 0.00 | 0.62 | 1.02 | 0.64 |
| BG in 70-180 | 97.29 ± | 90.13 ± | 98.71 ± | 98.07 ± | 96.33 ± |
| mg/dL [%] | 1.48 | 5.63 ( [symbol] ) | 0.97 | 0.96 | 2.29 (♦♦) |
| BG in 80-120 | 73.05 ± | 54.82 ± | 76.60 ± | 67.27 ± | 59.85 ± |
| mg/dL [%] | 5.93 | 8.66 ( [symbol] ) | 4.64 | 4.76 (♦) | 6.37 (♦♦) |
| BG > 180 | 2.71 ± | 9.87 ± | 0.60 ± | 0.67 ± | 3.00 ± |
| mg/dL [%] | 1.48 | 5.63 ( [symbol] ) | 0.61 | 0.66 | 2.34 (♦♦) |
| Median BG | 110.37 ± | 117.45 ± | 110.01 ± | 110.58 ± | 113.44 ± |
| [mg/dL] | 0.55 | 4.90 ( [symbol] ) | 0.32 | 1.26 | 3.57 (♦♦) |
| Minimum | 92.21 ± | 104.71 ± | 67.17 ± | 56.43 ± | 68.74 ± |
| BG [mg/dL] | 6.10 | 3.16 ( [symbol] ) | 13.08 | 12.98 | 15.81 |
| Maximum | 196.38 ± | 221.01 ± | 186.87 ± | 187.23 ± | 208.83 ± |
| BG [mg/dL] | 9.87 | 12.05 ( [symbol] ) | 9.68 | 12.61 | 13.62 (♦) |

Glucose metrics for nominal and robustness performance comparison of the proposed PID, a PID designed using an earlier, conventional model, and the proposed PID with model mismatch due to SC sensing (IP-SC). Note: Statistical significance is computed against the proposed PID (IP-IP) controller for nominal and robustness analysis using a Wilcoxon rank sum test. The symbol [symbol] implies p-values <0.05, respectively; comparison is between the proposed PID and the PID for the nominal case. The symbols ♦ and ♦♦ imply p-values <0.05 and <0.001, respectively; comparison is between the proposed PID, and both PID controllers in the last two columns of the table for the robustness case.

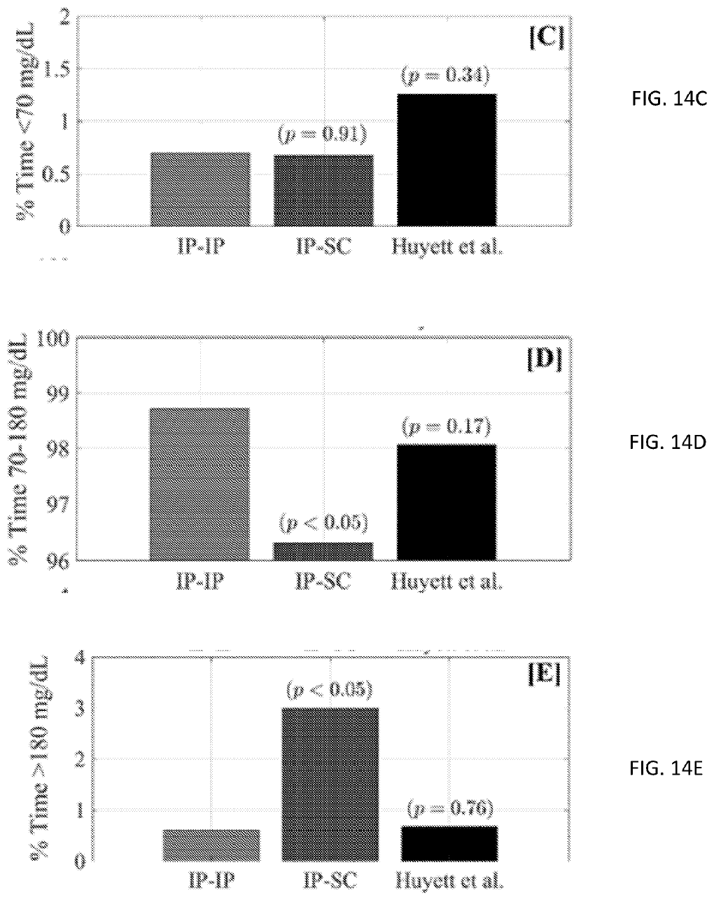

FIGS. 14A-14E demonstrate that the proposed controller is capable of handling unannounced meal challenges, sudden glucose declivity due to unannounced physical exercise, along with measurement noise and model mismatch. To this end, the closed-loop system is re-simulated using an SC sensor in the UVA/Padova metabolic simulator. Results from this evaluation are provided in FIGS. 14A-14E, with corresponding insulin-glucose values reported in the 3rd to 5th columns (titled “Robustness Analysis”) of Table 1, above.

It is clear from FIGS. 14A-14E that the presence of sensing lags and susceptibility to measurement noise produces a conservative control action (dotted line) trajectory compared to the IP-IP case (solid line). Hence, the time in the 70-180 mg/dL range is reduced (98.7±1.0% to 98.1±1.0%; p=0.91) and the time in severe hypoglycemia (<54 mg/dL) is slightly increased (0.1±0.3% to 0.5±0.7%; p=0.11) with the glucose minimum falling by ≈11 mg/dL (67.2±13.1 mg/dL to 56.4±13.0 mg/dL; p=0.85), although these differences are statistically indistinguishable. Both the time above 180 mg/dL (0.6±0.6% to 0.7±0.6%; p=0.76), and the maximum glucose level (186.9±9.7 mg/dL to 173.8±8.1 mg/dL; p=0.79) are similar for this cohort. As demonstrated in the nominal scenario, the proposed PID takes advantage of the fast clearance rate of insulin in the IP cavity and exhibits markedly improved control performance compared to the PID designed using an earlier IP-IP model (dashed line). Remarkable improvements are demonstrated in the tight 80-120 mg/dL range (76.6±4.6% to 59.9±6.4%; p<0.001) with the redesign, as is the time spent >180 mg/dL (0.6±0.6% to 3.0±2.3%; p<0.001). The improved quality of the control can also be attributed to the method of controller design: unlike the method of approximating higher-order transfer functions by lower-order ones described in previous designs, the full dynamical intricacies can be exhibited by the higher-order transfer function and FIGS. 14A-14E show the frequency response behavior in a bandwidth of interest. This leads to lesser degrees of approximation, and as evident by the simulation study, tighter closed-loop control.

### Experimental Data—Human Testing

The present disclosure can also provide for testing the proposed controller on humans. An exemplary experimental protocol can provide for ten adults (7 male, 3 female) with T1DM participating in a non-randomized, non-blinded sequential AP study using subcutaneous glucose sensing via Dexcom Seven Plus sensors (Dexcom, San Diego, USA) and zone MPC modified for IP delivery via the Diaport system (Second Generation, Roche Diagnostics, Mannheim, Germany). The clinical protocol can include three unannounced meals with 70, 40 and 70 g carbohydrate, respectively.

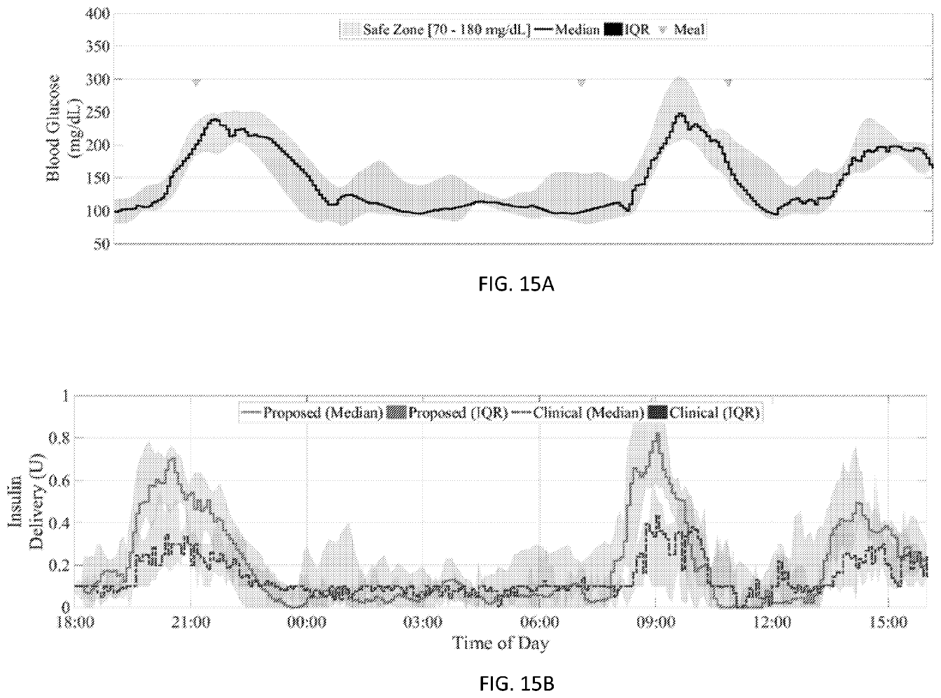

FIGS. 15A-15B show the behavior of the proposed PID controller on real patient data obtained from this clinical study. Although this test does not leverage feedback since the glucose values are used exactly as obtained in the clinical study, one can ascertain whether the controller performs expectedly, or whether the controller exhibits anomalous characteristics when faced with real sensor noise and glucose variability.

The median and interquartile range of BG for 10 patients is shown in FIG. 15A. FIG. 15B compares the control actions of the clinical controller zone MPC (dashed line) and the proposed PID controller (solid line). A Wilcoxon rank sum test reveals that the two control trajectories are not statistically significant (p=0.28), which is reflected by the similarity of the two control trajectories. Disparity between the two controllers can be observed at (i) around the meal disturbances, where the interquartile ranges imply that the proposed controller is more aggressive than the clinical controller; (ii) around 8 AM that the proposed controller is more sensitive to glucose excursions than the clinical controller; there is a 60 min delay before the zone MPC reacts to the glucose increase; and (iii) prolonged suspension after a meal compensating bolus. The speed of IP sensing and the faster poles of the new model enables the quick response of the proposed controller.

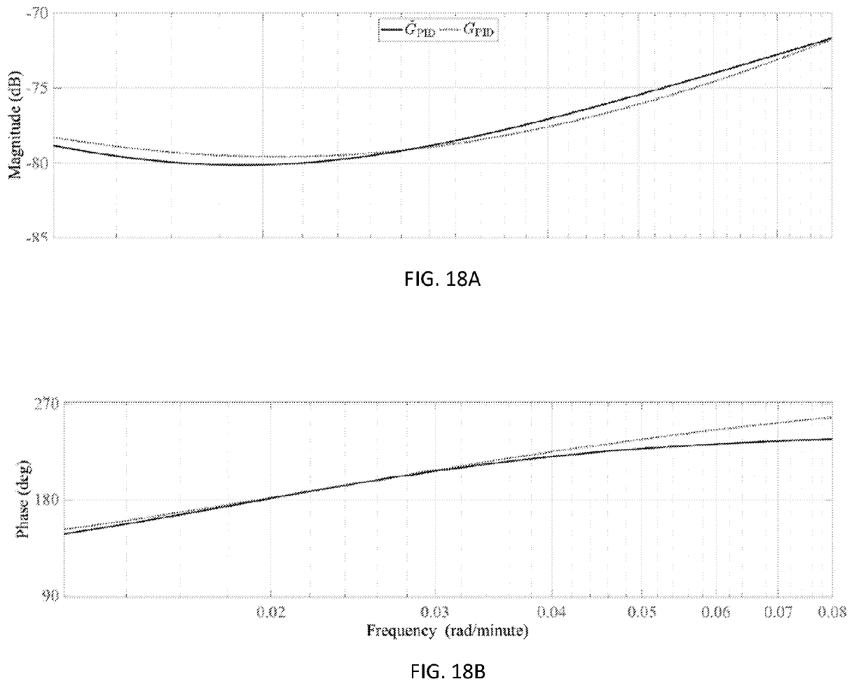

FIG. 18A shows exemplary data of the magnitude of transfer function matching via a convex optimization, according to an embodiment of the present disclosure. FIG. 18B shows exemplary data of the phase of transfer function matching via a convex optimization, according to an embodiment of the present disclosure. The solid lines denote frequency responses of the higher-order transfer function induced by internal model control tuning rules. The dashed line represents the best fit for the PID controller.

The proposed PID controller can be compared to a prior design where the control-relevant model was formulated using simulated data. The improvement of control performance in the IP space over the SC space is verified in these studies, both in-silico, and clinically. Herein, the proposed PID outperforms these algorithms due to a complete redesign of the controller using the new dynamical models.

### Analysis of Experimental Data

Therefore, FIGS. 3A-19B show that the experiments, according to an embodiment of the present disclosure, supported physiological knowledge that insulin clearance rates in the IP space are faster than SC space, and closed-loop numerical simulations illustrate potential of IP delivery in next-generation artificial pancreas (AP) systems.

An artificial pancreas system capable of leveraging the rapid sensing and actuation dynamics in the IP cavity shows can pave the way towards completely automated glucose management for type 1 diabetics. The present disclosure heads in this direction, where experimental data in the IP space is collected using canines, a species with insulin-glucose kinetics with close resemblance to humans. This data is utilized to formulate a control-relevant model that reflects these faster dynamics and can be used seamlessly in PID or model predictive controllers for AP research. The model is compared to prior models used in clinical studies via simulations and personalized for different people based on their total daily insulin intake. A PID controller designed using optimization-based transfer function matching (instead of empirical tuning rules) is subsequently constructed and tested on a challenging scenario with unannounced meals, sensor noise, and model mismatch.

The proposed closed-loop system operating in the IP space has several important advantages over conventional models. First, the proposed system reflects the faster insulin clearance kinetics that is expected in the IP cavity compared to the earlier model that is based on simulated data. This, in turn, enables the proposed PID controller to recommend more aggressive control strategies without producing sustained controller-induced hypoglycemia. This is especially important in vulnerable cohorts such as youth and adolescents with type 1 diabetes, where glucose variability is much higher than in adults, and maintaining tight glucose control (especially hypoglycemia prevention) is very challenging. Second, the proposed control architecture possesses very low complexity; indeed, the computational bottleneck lies in the computation of a simple controller update Equation 26 that involves a few simple arithmetical operations. This simplicity of execution implies that the proposed control architecture is amenable to implementation on resource-limited platforms such as implantable AP systems of the future. Third, the newly proposed controller tuning framework allows the user to design the controller gains using higher-order models of insulin-glucose dynamics. Generally, lower order models are used for controller design, which involves removal of higher-order poles and, therefore, loss of dynamical information. Preserving higher-order dynamics using the proposed approach contributes to the improvement of the controller performance.

Several challenges associated with fully-implantable IP insulin delivery systems warrant consideration, including risk of infection, insulin under-delivery related to insulin aggregation, and IP catheter obstruction. As experience with implantable IP insulin delivery has accumulated, however, the risk of these complications has decreased considerably. Further development is likely needed to miniaturize the pump device, maximize battery life, and extend the amount of time needed between insulin refill procedures before such systems are broadly accepted.

Despite extensive development over the past decade, fully-automatic closed-loop insulin delivery systems have yet to demonstrate comparable postprandial glycemic control compared with closed loop systems that employ meal announcement strategies. The favorable pharmacokinetic-pharmacodynamic profile associated with IP insulin delivery provides a practical approach for fully closed-loop systems to normalize postprandial hyperglycemia, a prominent contributor to overall hyperglycemia and cardiovascular complications in T1DM.

Moreover, a key limitation of current therapy is the necessity of insulin delivery into subcutaneous tissue. This approach requires higher levels of insulin in the peripheral circulation to provide the liver with an adequate amount of insulin to prevent excessive hepatic glucose production and hyperglycemia. Concurrently, a narrow therapeutic balance must be achieved where the higher insulin concentrations in the peripheral circulation do not lead to excessive muscle glucose uptake and hypoglycemia. By contrast, IP insulin delivery would be expected to restore the physiologic insulin distribution between the hepatic portal and peripheral circulations. Due to portal insulin absorption and first-pass hepatic insulin extraction, control of hepatic glucose production would not require the price of over-insulinization at muscle. Thus, when compared with subcutaneous closed-loop systems, the IP approach would operate on a flatter portion of the dose-response curve relating insulin dose to change in blood glucose. This factor that would mitigate the early postprandial hyperglycemia and late postprandial hypoglycemia associated with fully automated closed-loop systems presently, paving the way towards eliminating the burden of meal and exercise announcement and thereby improving quality of care and quality of life in people with T1DM.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### Conclusion

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

F. J. Doyle III, L. M. Huyett, J. B. Lee, H. C. Zisser, E. Dassau, Closed-Loop Artificial Pancreas Systems: Engineering the Algorithms, Diabetes Care. 37 (2014) 1191-1197. doi: 10.2337/dc13-2108.

A. Haidar, The Artificial Pancreas: How Closed-Loop Control Is Revolutionizing Diabetes, IEEE Control Syst. 36 (2016) 28-47. doi: 10.1109/MCS.2016.2584318.

R. Hovorka, Continuous glucose monitoring and closed-loop systems, Diabet. Med. 23 (2006) 1-12. doi:10.1111/j.1464-5491.2005.01672.x.

G. M. Steil, K. Rebrin, C. Darwin, F. Hariri, M. F. Saad, Feasibility of Automating Insulin Delivery for the Treatment of Type 1 Diabetes, Diabetes. 55 (2006) 3344-3350. doi:10.2337/db06-0419.

S. N. Mehta, N. Quinn, L. K. Volkening, L. M. B. Laffel, Impact of Carbohydrate Counting on Glycemic Control in Children With Type 1 Diabetes, Diabetes Care. 32 (2009) 1014-1016. doi:10.2337/dc08-2068.

K. J. Bell, C. E. Smart, G. M. Steil, J. C. Brand-Miller, B. King, H. A. Wolpert, Impact of Fat, Protein, and Glycemic Index on Postprandial Glucose Control in Type 1 Diabetes: Implications for Intensive Diabetes Management in the Continuous Glucose Monitoring Era, Diabetes Care. 38 (2015) 1008-1015. doi:10.2337/dc15-0100.

E. Renard, G. Costalat, H. Chevassus, J. Bringer, Artificial beta-cell: clinical experience toward an implantable closed-loop insulin delivery system., Diabetes Metab. 32 (2006) 497-502. http://www.ncbi.nlm.nih.gov/pubmed/17130808.

E. Renard, J. Place, M. Cantwell, H. Chevassus, C. C. Palerm, Closed-loop insulin delivery using a subcutaneous glucose sensor and intraperitoneal insulin delivery: feasibility study testing a new model for the artificial pancreas., Diabetes Care. 33 (2010) 121-7. doi:10.2337/dc09-1080.

A. Liebl, R. Hoogma, E. Renard, P. H. L. M. Geelhoed-Duijvestijn, E. Klein, J. Diglas, L. Kessler, V. Melki, P. Diem, 560 J.-M. Brun, P. Schaepelynck-Bélicar, T. Frei, European DiaPort Study Group, A reduction in severe hypoglycaemia in type 1 diabetes in a randomized crossover study of continuous intraperitoneal compared with subcutaneous insulin infusion., Diabetes. Obes. Metab. 11 (2009) 1001-8. doi:10.1111/j.1463-1326.2009.01059.x.

E. Dassau, E. Renard, J. Place, A. Farret, M. Pelletier, J. Lee, L. M. Huyett, A. Chakrabarty, F. J. Doyle III, H. C. Zisser, Intraperitoneal Insulin Delivery Provides Superior Glycemic Regulation to Subcutaneous Insulin Delivery in Model Predictive Control-based Fully-automated Artificial Pancreas in Patients with Type 1 Diabetes: A Pilot Study, Diabetes, Obes. Metab. (2017).

R. P. Eaton, R. C. Allen, D. S. Schade, Hepatic removal of insulin in normal man: dose response to endogenous insulin secretion., J. Clin. Endocrinol. Metab. 56 (1983) 1294 300. doi:10.1210/jcem-56-6-1294.

D. L. Horwitz, J. I. Starr, M. E. Mako, W. G. Blackard, A. H. Rubenstein, Proinsulin, insulin, and C-peptide concentrations in human portal and peripheral blood., J. Clin. Invest. 55 (1975) 1278-83. doi:10.1172/JCI108047.

J. M. Gregory, G. Kraft, M. F. Scott, D. W. Neal, B. Farmer, M. S. Smith, J. R. Hastings, E. J. Allen, E. P. Donahue, N. Rivera, J. J. Winnick, D. S. Edgerton, E. Nishimura, C. Fledelius, C. L. Brand, A. D. Cherrington, Insulin Delivery Into the Peripheral Circulation: A Key Contributor to Hypoglycemia in Type 1 Diabetes., Diabetes. 64 (2015) 3439-51. doi:10.2337/db15-0071.

S. Del Prato, F. Leonetti, D. C. Simonson, P. Sheehan, M. Matsuda, R. A. DeFronzo, Effect of sustained physiologic hyperinsulinaemia and hyperglycaemia on insulin secretion and insulin sensitivity in man., Diabetologia. 37 (1994) 1025-35. http://www.ncbi.nlm.nih.gov/pubmed/7851681.

H.-Y. Liu, S. Y. Cao, T. Hong, J. Han, Z. Liu, W. Cao, Insulin is a stronger inducer of insulin resistance than hyperglycemia in mice with type 1 diabetes mellitus (T1DM)., J. Biol. Chem. 284 (2009) 27090-100. doi:10.1074/jbc.M109.016675.

P. R. van Dijk, K. H. Groenier, J. H. DeVries, R. O. B. Gans, N. Kleefstra, H. J. G. Bilo, S. J. J. Logtenberg, Continuous intraperitoneal insulin infusion versus subcutaneous insulin therapy in the treatment of type 1 diabetes: effects on glycemic variability., Diabetes Technol. Ther. 17 (2015) 379-84. doi:10.1089/dia.2015.0001.

P. R. van Dijk, S. J. J. Logtenberg, S. I. Chisalita, C. A. Hedman, K. H. Groenier, R. O. B. Gans, N. Kleefstra, H. J. Arnqvist, H. J. G. Bilo, Different Effects of Intraperitoneal and Subcutaneous Insulin Administration on the GH-IGF-1 Axis in Type 1 Diabetes., J. Clin. Endocrinol. Metab. 101 (2016) 2493-501. doi:10.1210/jc.2016-1473.

P. R. van Dijk, S. J. J. Logtenberg, R. O. B. Gans, H. J. G. Bilo, N. Kleefstra, Intraperitoneal insulin infusion: treatment option for type 1 diabetes resulting in beneficial endocrine effects beyond glycaemia., Clin. Endocrinol. (Oxf). 81 (2014) 488-97. doi:10.1111/cen.12546.

D. R. Burnett, L. M. Huyett, H. C. Zisser, F. J. Doyle III, B. D. Mensh, Glucose sensing in the peritoneal space offers faster kinetics than sensing in the subcutaneous space., Diabetes. 63 (2014) 2498-505. doi:10.2337/db13-1649.

L. M. Huyett, R. Mittal, H. C. Zisser, E. S. Luxon, A. Yee, E. Dassau, F. J. Doyle III, D. R. Burnett, Preliminary Evaluation of a Long-Term Intraperitoneal Glucose Sensor With Flushing Mechanism., J. Diabetes Sci. Technol. 10 (2016) 1192-4. doi:10.1177/1932296816640542.

A. L. Fougner, K. Kolle, N. K. Skjarvold, N.-A. L. Elvemo, D. R. Hjelme, R. Ellingsen, S. M. Carlsen, Ø. Stavdahl, Intraperitoneal Glucose Sensing is Sometimes Surprisingly Rapid, Model. Identif. Control A Nor. Res. Bull. 37 (2016) 121-131. doi:10.4173/mic.2016.2.4.

S. Zavitsanou, A. Chakrabarty, E. Dassau, F. J. Doyle III, Embedded Control in Wearable Medical Devices: Application to the Artificial Pancreas, Processes. 4 (2016) 35.

A. Chakrabarty, S. Zavitsanou, F. J. Doyle III, E. Dassau, Event-Triggered Model Predictive Control for Embedded Artificial Pancreas Systems, IEEE Trans. Biomed. Eng. 65 (2018) 575-586. doi:10.1109/TBME.2017.2707344.

S. R. Myers, D. W. Biggers, D. W. Neal, A. D. Cherrington, Intraportal glucose delivery enhances the effects of hepatic glucose load on net hepatic glucose uptake in vivo., J. Clin. Invest. 88 (1991) 158-67. doi:10.1172/JCI115273.

R. L. Dobbins, S. N. Davis, D. W. Neal, C. Cobelli, J. Jaspan, A. D. Cherrington, Compartmental modeling of glucagon kinetics in the conscious dog., Metabolism. 44 (1995) 452-9. http://www.ncbi.nlm.nih.gov/pubmed/7723667.

S. S. Yen, T. M. Siler, G. W. DeVane, Effect of somatostatin in patients with acromegaly: suppression of growth 612hormone, prolactin, insulin and glucose levels., N. Engl. J. Med. 290 (1974) 935-8. doi:10.1056/NEJM197404252901704.

D. J. Koerker, W. Ruch, E. Chideckel, J. Palmer, C. J. Goodner, J. Ensinck, C. C. Gale, Somatostatin: hypothalamic inhibitor of the endocrine pancreas., Science. 184 (1974) 482 4. http://www.ncbi.nlm.nih.gov/pubmed/4594711.

J. C. Reubi, L. Kvols, E. Krenning, S. W. Lamberts, Distribution of somatostatin receptors in normal and tumor tissue., Metabolism. 39 (1990) 78-81. http://www.ncbi.nlm.nih.gov/pubmed/2169575.

A. D. Cherrington, J. L. Chiasson, J. E. Liljenquist, A. S. Jennings, U. Keller, W. W. Lacy, The role of insulin and 620 glucagon in the regulation of basal glucose production in the postabsorptive dog., J. Clin. Invest. 58 (1976) 1407-18. doi:10.1172/JCI108596.

D. S. Edgerton, S. Cardin, M. Emshwiller, D. Neal, V. Chandramouli, W. C. Schumann, B. R. Landau, L. Rossetti, A. D. Cherrington, Small increases in insulin inhibit hepatic glucose production solely caused by an effect on glycogen metabolism., Diabetes. 50 (2001) 1872-82. http://www.ncbi.nlm.nih.gov/pubmed/11473051.

L. M. Huyett, E. Dassau, H. C. Zisser, F. J. Doyle III, Design and Evaluation of a Robust PID Controller for a Fully Implantable Artificial Pancreas, Ind. Eng. Chem. Res. 54 (2015) 10311-10321. doi:10.1021/acs.iecr.5b01237.

J. J. Lee, E. Dassau, H. Zisser, F. J. Doyle III, Design and in silico evaluation of an intraperitoneal-subcutaneous (IP-SC)artificial pancreas., Comput. Chem. Eng. 70 (2014) 180-188. doi:10.1016/j.compchemeng.2014.02.024.

K. van Heusden, E. Dassau, H. C. Zisser, D. E. Seborg, F. J. Doyle III, Control-Relevant Models for Glucose Control Using A Priori Patient Characteristics, IEEE Trans. Biomed. Eng. 59 (2012) 1839-1849. doi:10.1109/TBME.2011.2176939.

B. P. Kovatchev, M. Breton, C. Dalla Man, C. Cobelli, In silico preclinical trials: a proof of concept in closed-loop control of type 1 diabetes., J. Diabetes Sci. Technol. 3 (2009) 44-55. doi:10.1177/193229680900300106.

F. N. Fritsch, R. E. Carlson, Monotone Piecewise Cubic Interpolation, SIAM J. Numer. Anal. 17 (1980) 238-246. doi:10.1137/0717021.

D. E. Rivera, M. Moran, S. Skogestad, Internal model control: PID controller design, Ind. Eng. Chem. Process Des. Dev. 25 (1986) 252-265. doi:10.1021/i200032a041.

B. W. Bequette, Process control: modeling, design, and simulation, First, Prentice Hall, Upper Saddle River, N.J., 2003.

D. Seborg, T. Edgar, D. Mellicamp, F. Doyle III, Process Dynamics and Control, 2011. doi:10.1007/s13398-014-0173-7.2.

C. C. Palerm, Physiologic insulin delivery with insulin feedback: A control systems perspective, Comput. Methods Programs Biomed. 102 (2011) 130-137. doi: 10.1016/j.cmpb.2010.06.007.

P. Soon-Shiong, E. Feldman, R. Nelson, J. Komtebedde, O. Smidsrod, G. Skjak-Braek, T. Espevik, R. Heintz, M. Lee, Successful reversal of spontaneous diabetes in dogs by intraperitoneal microencapsulated islets., Transplantation. 54 (1992) 769-74. http://www.ncbi.nlm.nih.gov/pubmed/1440841.

Y. Matsuo, S. Shimoda, M. Sakakida, K. Nishida, T. Sekigami, S. Ichimori, K. Ichinose, M. Shichiri, E. Araki, Strict glycemic control in diabetic dogs with closed-loop intraperitoneal insulin infusion algorithm designed for an artificial endocrine pancreas., J. Artif. Organs. 6 (2003) 55-63. doi:10.1007/s100470300009.

A. E. Panteleon, M. Loutseiko, G. M. Steil, K. Rebrin, Evaluation of the effect of gain on the meal response of an automated closed-loop insulin delivery system., Diabetes. 55 (2006) 1995-2000. doi:10.2337/db05-1346.

A. D. Cherrington, Control of glucose uptake and release by the liver in vivo., Diabetes. 48 (1999) 1198-214. http://www.ncbi.nlm.nih.gov/pubmed/10331429.

M. Scavini, A. Pincelli, G. Petrella, G. Galimberti, P. G. Zager, M. Torri, G. Pozza, Intraperitoneal insulin absorption after long-term intraperitoneal insulin therapy., Diabetes Care. 18 (1995) 56-9. http://www.ncbi.nlm.nih.gov/pubmed/7698048.

M. Seita, H. Noguchi, Y. Kubota, H. Kawamoto, S. Nakaji, N. Kobayashi, T. Fujiwara, Development of Canine Models of Type 1 Diabetes With Partial Pancreatectomy and the Administration of Streptozotocin., Cell Med. 6 (2013) 25-31. doi:10.3727/215517913X674289.

S. R. Colberg, J. Walsh, Pumping insulin during exercise: What healthcare providers and diabetic patients need to know, Phys. Sportsmed. 30 (2002) 33-38. doi:10.3810/psm.2002.04.255.

A. E. Panteleon, E. Renard, J. Han, P. Leong, M. K. Kerstin Rebrin, G. M. Steil, Quantification of Delays Associated with Intraperitoneal Insulin Delivery and IV Glucose Sensing Aiming at Closed Loop Insulin Delivery., Diabetes. 53 (2004) A105.

G. M. Steil, M. F. Saad, Automated insulin delivery for type 1 diabetes, Curr. Opin. Endocrinol. Diabetes. 13 (2006) 205-211. doi:10.1097/01.med.0000216971.61160.57.

A. L. O'Kell, T. J. Garrett, C. Wasserfall, M. A. Atkinson, Untargeted metabolomic analysis in naturally occurring canine diabetes mellitus identifies similarities to human Type 1 Diabetes., Sci. Rep. 7 (2017) 9467. doi:10.1038/s41598-017-09908-5.

A. Tvarijonaviciute, J. J. Ceron, S. L. Holden, D. J. Cuthbertson, V. Biourge, P. J. Morris, A. J. German, Obesity-related metabolic dysfunction in dogs: a comparison with human metabolic syndrome., BMC Vet. Res. 8 (2012) 147. doi:10.1186/1746-6148-8-147.

T. T. Ly, D. B. Keenan, A. Roy, J. Han, B. Grosman, M. Cantwell, N. Kurtz, R. von Eyben, P. Clinton, D. M. Wilson, B. A. Buckingham, Automated Overnight Closed-Loop Control Using a Proportional-Integral-Derivative Algorithm with Insulin Feedback in Children and Adolescents with Type 1 Diabetes at Diabetes Camp, Diabetes Technol. Ther. 18 (2016) 377-384. doi:10.1089/dia.2015.0431.

E. Renard, J. Bringer, D. Jacques-Apostol, D. Lauton, C. Mestre, G. Costalat, C. Jaffiol, Complications of the pump pocket may represent a significant cause of incidents with implanted systems for intraperitoneal insulin delivery, Diabetes Care. 17 (1994) 1064-1066. doi:10.2337/diacare.17.9.1064.

E. Renard, S. Bouteleau, D. Jacques-Apostol, D. Lauton, F. Gibert-Boulet, G. Costalat, J. Bringer, C. Jaffiol, Insulin underdelivery from implanted pumps using peritoneal route. Determinant role of insulin pump compatibility., Diabetes Care. 19 (1996) 812-7. http://www.ncbi.nlm.nih.gov/pubmed/8842596.

N. A. Spaan, A. E. Teplova, E. Renard, J. A. E. Spaan, Implantable insulin pumps: an effective option with restricted dissemination., Lancet. Diabetes Endocrinol. 2 (2014) 358-60. doi:10.1016/S2213-8587 (14) 70035-X.

P. Schaepelynck, E. Renard, N. Jeandidier, H. Hanaire, C. Fermon, S. Rudoni, B. Catargi, J.-P. Riveline, B. Guerci, L. Millot, J.-F. Martin, A. Sola, EVADIAC Group, A recent survey confirms the efficacy and the safety of implanted insulin pumps during long-term use in poorly controlled type 1 diabetes patients., Diabetes Technol. Ther. 13 (2011) 657-60. doi:10.1089/dia.2010.0209.

E. Renard, B. Guerci, A.-M. Leguerrier, R. Boizel, Accu-Chek FlexLink Study Group, Lower rate of initial failures and reduced occurrence of adverse events with a new catheter model for continuous subcutaneous insulin infusion: prospective, two-period, observational, multicenter study., Diabetes Technol. Ther. 12 (2010) 769-73. doi:10.1089/dia.2010.0073.

H. Gin, E. Renard, V. Melki, S. Boivin, P. Schaepelynck-Bélicar, B. Guerci, J. L. Selam, J. M. Brun, J. P. Riveline, B. Estour, B. Catargi, EVADIAC Study Group, Combined improvements in implantable pump technology and insulin stability allow safe and effective long term intraperitoneal insulin delivery in type 1 diabetic patients: the EVADIAC experience., Diabetes Metab. 29 (2003) 602-7. http://www.ncbi.nlm.nih.gov/pubmed/14707889.

C. L. Rohlfing, H.-M. Wiedmeyer, R. R. Little, J. D. England, A. Tennill, D. E. Goldstein, Defining the relationship between plasma glucose and HbA(1c): analysis of glucose profiles and HbA(1c) in the Diabetes Control and Complications Trial., Diabetes Care. 25 (2002) 275-8. http://www.ncbi.nlm.nih.gov/pubmed/11815495.

A. Ceriello, Postprandial Glucose Regulation and Diabetic Complications, Arch. Intern. Med. 164 (2004) 2090. doi:10.1001/archinte.164.19.2090.

J. L. Ruiz, J. L. Sherr, E. Cengiz, L. Carria, A. Roy, G. Voskanyan, W. V Tamborlane, S. A. Weinzimer, Effect of insulin feedback on closed-loop glucose control: a crossover study., J. Diabetes Sci. Technol. 6 (2012) 1123-30. doi:10.1177/193229681200600517.

A. C. van Bon, Y. M. Luijf, R. Koebrugge, R. Koops, J. B. L. Hoekstra, J. H. DeVries, Feasibility of a portable bihormonal closed-loop system to control glucose excursions at home under free-living conditions for 48 hours., Diabetes Technol. Ther. 16 (2014) 131-6. doi:10.1089/dia.2013.0166.

1. A system for maintaining glycemic control of a patient, comprising:
at least one sensor configured to detect glucose in an intraperitoneal space of the patient;
an insulin infusion pump configured to inject insulin into the intraperitoneal space; and
a glucose monitoring system, wherein the glucose monitoring system is configured to:
receive data from the at least one sensor; and
send instructions to the insulin infusion pump, wherein the instructions are based on a general control algorithm and the received data.

2. The system of claim 1, wherein the general control algorithm comprises a closed-loop proportional-integral derivative (PID) algorithm, the closed-loop PID control algorithm being an optimization-based transfer function matching method.

3. (canceled)

4. The system of claim 1, wherein the general control algorithm comprises a closed-loop proportional-integral derivative (PID) algorithm, the closed-loop PID control algorithm being a discrete-time transfer function model.

5. The system of claim 4, wherein the discrete-time transfer function model compensates for steady-state gain contributed by poles of the discrete-time transfer function model.

6. The system of claim 4, wherein the discrete-time transfer function model includes a total daily insulin intake for the patient.

7. The system of claim 1, wherein the general control algorithm comprises a closed-loop proportional-integral derivative (PID) algorithm and the glucose monitoring system is further configured to interpolate the received data using a piecewise Hermite or Legendre polynomial interpolation scheme.

8. (canceled)

9. The system of claim 1, wherein the general control algorithm comprises a closed-loop proportional-integral derivative (PID) algorithm and the glucose monitoring system obtains at least one constant of the closed-loop PID control algorithm using discrete-time internal model control and optimization-based transfer function matching.

10. A method for maintaining glycemic control of a patient, comprising:
receiving glucose data from at least one sensor in an intraperitoneal space of the patient;
processing the received glucose data at a glucose monitoring system to yield processed data; and instructing, by the glucose monitoring system, an insulin infusion pump, wherein the instructing is based on a general control algorithm and the processed data.

11. The method of claim 10, wherein the general control algorithm comprises a closed-loop PID control algorithm, the closed-loop PID control algorithm being an optimization-based transfer function matching method.

12. (canceled)

13. The method of claim 10, wherein the general control algorithm comprises a closed-loop PID control algorithm, the closed-loop PID control algorithm comprises being a discrete-time transfer function model.

14. The method of claim 13, wherein the discrete-time transfer function model determines a steady-state gain of a plurality of poles of the discrete-time transfer function model.

15. The method of claim 13, wherein the discrete-time transfer function model is based on a total daily insulin intake for the patient.

16. The method of claim 10, wherein the general control algorithm comprises a closed-loop PID control algorithm and the processing further comprises interpolating the received glucose data using a piecewise Hermite polynomial interpolation scheme.

17. (canceled)

18. The method of claim 10, wherein the general control algorithm comprises a closed-loop PID control algorithm and the glucose monitoring system obtains at least one constant of the closed-loop PID control algorithm using discrete-time internal model control and optimization-based transfer function matching.

19. A non-transitory machine readable medium having stored thereon instructions for performing a method comprising machine executable code which when executed by at least one machine, causes the machine to:
receive glucose data from at least one sensor;
process the received glucose data at a glucose monitoring system to yield processed data; and
instruct, by the glucose monitoring system, an insulin infusion pump, wherein the instructing is based on a closed-loop proportional-integral derivative (PID) algorithm and the processed data.

20. (canceled)

21. The non-transitory machine readable medium of claim 19, wherein the closed-loop PID control algorithm comprises an optimization-based transfer function matching method.

22. The non-transitory machine readable medium of claim 19, wherein: the closed-loop PID control algorithm comprises a discrete-time transfer function model and the discrete-time transfer function model determines a steady-state gain of a plurality of poles of the discrete-time transfer function model.

23. (canceled)

24. The non-transitory machine readable medium of claim 19, wherein: the closed-loop PID control algorithm comprises a discrete-time transfer function model;
and the discrete-time transfer function model is based on a total daily insulin intake for the patient.

25. The non-transitory machine readable medium of claim 19, wherein the processing further comprises interpolating the received glucose data using a piecewise polynomial interpolation scheme.

26. (canceled)

27. The non-transitory machine readable medium of claim 19, wherein the glucose monitoring system obtains at least one constant of the closed-loop PID control algorithm using discrete-time internal model control and optimization-based transfer function matching.
